(19) United States
(12) Patent Application Publication
(10) Pub. No.: US 2005/0288212 A1
(43) Pub. Date: Dec. 29, 2005

(54) Components of the presenilin-complex

(75) Inventors: Richard Hale, Prestwood (GB); Adele Rowley, St Albans (GB)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: CELLZOME AG, Heidelberg (DE)

(21) Appl. No.: 10/519,238

(22) PCT Filed: Jun. 25, 2003
(86) PCT No.: PCT/EP03/06704

§ 371 Date: Aug. 1, 2005

(30) Foreign Application Priority Data
Jun. 26, 2002 (EP) 02014244.4

(51) Int. Cl. A61K 38/17; G01N 33/53; G01N 33/567; C07K 14/705; C07K 16/28

(52) U.S. Cl. 514/2; 435/7.2; 530/350; 530/388.1

(57) ABSTRACT

The present invention is based on a novel direct interaction between a Presenilin and a novel protein identified herein and named Sambiasin-1, a homolog thereof named Sambiasin-2, as well as a protein complex further comprising a Nicastrin. Also comprised are uses of said components and complexes, as well as methods for use of the protein and the complex, inter alia, screening, diagnosis and therapy, as well as methods of preparing the complexes.

### 1. FIELD OF THE INVENTION

The present invention relates to components of the Presenilin-complex, fragments and derivatives of the component proteinsx, the complete protein complex, uses of said components and complex as well as methods for use of the protein and the complex, inter alia, screening, diagnosis, and therapy, as well as to methods of preparing the complexes.

### 2. BACKGROUND OF THE INVENTION

Alzheimer's disease is a chronic condition that affects millions of individuals worldwide. After onset of the disease sufferers require a high degree of supervision and care. As the proportion of aged individuals in the population increases, the number of sufferers of Alzheimer's disease is expected to expand dramatically. Current therapies treat symptoms of the disease and have limited success in the clinic. There are currently no therapies available that halt disease progression.

The brains of sufferers of Alzheimer's disease show a characteristic pathology of prominent neuropathologic lesions, such as neurofibrillary tangles (NFTs) and amyloid-rich senile plaques. These lesions are associated with massive loss of populations of CNS neurons and their development often accompanies the clinical dementia associated with AD. A major component of amyloid plaques is the amyloid beta peptide. Amyloid beta is the product of a precursor protein, beta amyloid precursor protein (b-APP). b-APP is a type-I trans-membrane protein which is cleaved by several different membrane-associated proteases. The first cleavage of b-APP occurs extracellularly by one of two proteases, alpha-secretase or beta-secretase. Beta-secretase or BACE (beta-site APP-cleaving enzyme) is a type-I transmembrane protein containing an aspartyl protease activity. Alpha secretase is a metalloprotease whose activity is most likely to be provided by one or a combination of the proteins ADAM10 and 17. Following either the beta or alpha cleavage of b-APP, the final cleavage event occurs within the membrane and is carried out by gamma secretase. It is the combination of the beta and gamma secretase activities that results in the liberation of the Abeta peptide from the b-APP and ultimately the formation of the amyloid plaques responsible for the pathology of Alzheimer's disease.

The identity of the gamma secretase cleaving activity remains elusive. There is a large body of evidence to suggest that the presenilin 1 and 2 proteins are intimately linked with the function of the gamma secretase. However, there is no evidence that these proteins alone constitute the gamma catalytic activity. Recent data suggests that the gamma secretase may not be derived from a single gene product, but that a large multimeric complex of proteins gives rise to the proteolytic activity. A number of proteins have been shown to reside in a complex with presenilin (PS) 1. Of these a more recent addition is the protein Nicastrin (WO-01/67109). Nicastrin has been shown to be part of a high molecular weight complex that can be co-purified with the presenilins and b-APP either by immunoprecipitation with anti-PS antisera or by affinity chromatography with a specific gamma secretase inhibitor (Yu et al, 2000; Esler et al, 2002). Together, the evidence points to Nicastrin playing an important role in gamma secretase cleavage of substrates.

In addition to cleaving b-APP, gamma secretase cleaves the protein Notch in a similar manner. Notch is involved in cell fate determination. The Notch gamma cleavage event is similar to that of b-APP in that the cleavage is the final in a series of proteolytic activities and occurs within the membrane. Both the presenilins and Nicastrin have been shown to be required for Notch cleavage (Yu et al, 2000). The C. elegans orthologue of Nicastrin, APH-2, is essential for Notch signaling during early embryogenesis.

Despite the large body of information already available from the prior art concerning presenilin proteins, up to now the picture of presenilin-interactor proteins remains elusive.

As a knowledge of which proteins associate with presenilin is of fundamental importance for the development of new therapies, an object of the present invention was to identify and provide new interactors of Presenilin and to provide new targets for therapy.

Said object is achieved by the Sambiasin according to the present invention and the complexes comprising the same.

### 3. SUMMARY OF THE INVENTION

The present invention is based a novel direct interaction between a presenilin and a novel protein identified herein and herein named Sambiasin-1 and a homolog thereof herein named Sambiasin-2 as well as a protein complex further comprising a nicastrin.

1. A protein complex comprising
(a) a first protein, or a functionally active fragment or functionally active derivative thereof, which first protein is selected from the group consisting of:
   (i) Sambiasin-1 (SEQ ID No: 1) or a functionally active derivative thereof, or a functionally active fragment thereof, or a homolog thereof, or a variant of Sambiasin-1 encoded by a nucleic acid that hybridizes to the Sambiasin-1 nucleic acid or its complement under low stringency conditions,
(b) a second protein, or a functionally active fragment or functionally active derivative thereof, which second protein is selected from the group consisting of:
   (i) Presenilin-1 (SEQ ID No: 2), or a functionally active derivative thereof, or a functionally active fragment thereof, or a homolog thereof, or a variant of Presenilin-1 encoded by a nucleic acid that hybridizes to the Presenilin-1 nucleic acid or its complement under low stringency conditions,
   (ii) Nicastrin (SEQ ID No: 3), or a functionally active derivative thereof, or a functionally active fragment thereof, or a homolog thereof, or a variant of Nicastrin encoded by a nucleic acid that hybridizes to the Nicastrin nucleic acid or its complement under low stringency conditions, wherein said first protein and said second protein are members of a native cellular complex, and wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at60° C.

2. A protein complex comprising Sambiasin-1 (SEQ ID No: 1) or Sambiasin-2 (SEQ ID No: 4) and Presenilin1 (SEQ ID No: 2) or Presenilin-2 (SEQ ID No: 5).

3. A protein complex according to No. 1,2 further comprising Nicastrin (SEQ ID No: 3)

4. A protein complex comprising Sambiasin-1 SEQ ID No: 1) and Presenilin-1 (SEQ ID No: 2) and Nicastrin (SEQ ID No: 3).

5. The complex of No. 1,2,3,4 comprising a functionally active derivative of any of the proteins of said complex, wherein the functionally active derivative is a fusion protein comprising said protein fused to an amino acid sequence different from said protein.

6. The complex of claim 5 wherein the functionally active derivative is a fusion protein comprising said protein fused to an affinity tag or label.

7. The complex of No. 1,2,3,4 comprising a fragment of any of the proteins of said complex, which fragment binds to another protein component of said complex.

8. The complex of No. 1,2,3,4,5,6,7 that is involved in the gamma-secretase activity.

9. Protein comprising the amino acid sequence of SEQ ID No: 1, or a functionally active derivative thereof, or a functionally active fragment thereof, or a homolog thereof, or a variant of Sambiasin-1 encoded by a nucleic acid that hybridizes to the Sambiasin-1 nucleic acid or its complement under low stringency conditions, wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C., with the provisio that the protein does not have the amino acid sequence according to SEQ ID 6.

10. Protein comprising the amino acid sequence of SEQ ID No: 1.

11. Nucleic acid encoding a protein according to No 9 or 10.

12. Construct, preferably a vector construct, comprising
(a) a nucleic acid according to No. 11 and at least one further nucleic acid which is normally not associated with said nucleic acid, or
(b) at least two separate nucleic acid sequences each encoding a different protein of any of the proteins, or a functionally active fragment or a functionally active derivative thereof according to No. 1.

13. Host cell, containing a vector comprising at least one of the nucleic acid of No. 11 and/or any of the constructs of No. 12 or containing several vectors each comprising at least the nucleic acid sequence encoding at least one of the proteins, or functionally active fragments or functionally active derivatives thereof selected from the first group of proteins according to No. 1.

14. An antibody or a fragment of said antibody containing the binding domain thereof, which binds the complex of any No. 1 to 8 and which does not bind the first protein when uncomplexed or the second protein when uncomplexed and/or an antibody or a fragment of said antibody containing the binding domain thereof which binds to any of the group of proteins according to any of No. 9, 10.

15. A kit comprising in one or more container the complex of any of No. 1 to 8 and/or the proteins of any of No. 9, 10, optionally together with an antibody according to No. 14 and/or further components such as reagents and working instructions.

16. A kit according to No. 15 for the diagnosis or prognosis of a disease or a disease risk, preferentially for a disease or disorder such as neurodegenerative diseases such as Alzheimer and developmental disorders caused by defects in the Notch pathway.

17. Array, in which at least a complex according to any of No. 1 to 8 and/or any of the proteins of any of No. 9 or 10 and/or at least one antibody according to No. 14 is attached to a solid carrier.

18. A process for processing the physiological substrates of any of the complexes of any of No. 1,2,3,4 comprising the step of bringing into contact a complex of any of No. 1 to 7 with said substrate, such that said substrate is processed.

19. A pharmaceutical composition comprising the protein complex of No. 1,2,3,4,5,6,7 or 8 and a pharmaceutically acceptable carrier and/or any of the proteins of No. 9 or 10 and a pharmaceutically acceptable carrier.

20. A pharmaceutical composition according to No. 19 for the treatment of diseases and disorders such as neurodegenerative diseases, such as Alzheimer, and/or developmental disorders caused by defects in the Notch pathway.

21. A method for screening for a molecule that binds to the complex of anyone of No. 1 to 8 and/or any of the proteins of No. 9 or 10, comprising the following steps:
(a) exposing said complex or protein, or a cell or organism containing same, to one or more candidate molecules; and
(b) determining whether said candidate molecule is bound to the complex or protein.

22. A method for screening for a molecule that modulates directly or indirectly the function, activity, composition or formation of the complex of any one of No. 1 to 8 comprising the steps of:
(a) exposing said complex, or a cell or organism containing said complex to one or more candidate molecules; and
(b) determining the amount of, activity of, protein components of, and/or intracellular localization of, said complex and/or the transcription level of a gene dependend on the complex and/or the abundance and/or activity of a protein or protein complex dependend on the function of the complex and/or product of a gene dependent on the complex in the presence of the one or more candidate molecules, wherein a change in said amount, activity, protein components or intracellular localization relative to said amount, activity, protein components and/or intracellular localization and/or a change in the transcription level of a gene dependend on the complex and/or the abundance and/or activity of a protein or protein complex dependent on the function of the complex and/or product of a gene dependent on the complex in the absence of said candidate molecules indicates that the molecule modulates function, activity or composition of said complex.

23. The method of No. 22 wherein the amount of said complex is determined.

24. The method of No. 22, wherein the activity of said complex is determined.

25. The method of No. 24, wherein said determining step comprises isolating from the cell or organism said complex to produce said isolated complex and contacting said isolated complex in the presence or absence of a candidate molecule with a physiological substrate of any of the complexes according to No. 1,2,3,4 and determine whether said substrate is processed.

26. The method of No. 22, wherein the amount of the individual protein components of said complex are determined.

27. The method of No. 26, wherein said determining step comprises determining whether
(i) Sambiasin-1 (SEQ ID No: 1), or a functionally active derivative thereof, or a functionally active fragment thereof, or a homolog thereof, or a variant of Sambiasin-1 encoded by a nucleic acid that hybridizes to the Sambiasin-1 nucleic acid or its complement under low stringency conditions, and/or
(ii) Presenilin-1 (SEQ ID No: 2), or a functionally active derivative thereof, or a functionally active fragment thereof, or a homolog thereof, or a variant of Presenilin-1 encoded by a nucleic acid that hybridizes to the Presenilin-1 nucleic acid or its complement under low stringency conditions, and/or
(iii) Nicastrin (SEQ ID No: 3), or a functionally active derivative thereof, or a functionally active fragment thereof, or a homolog thereof, or a variant of Nicastrin encoded by a nucleic acid that hybridizes to the Nicastrin nucleic acid or its complement under low stringency conditions,
   are present in the complex and wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.

28. The method of any of No. 22 to 27, wherein said method is a method of screening for a drug for treatment or prevention of a disease or disorder such as neurodegenerative diseases such as Alzheimer and developmental disorders caused by defects in the Notch pathway.

29. Use of a molecule that modulates the amount of, activity of, or the protein components of the complex of any one of No. 1 to 8 for the manufacture of a medicament for the treatment or prevention of a disease or disorder such as neurodegenerative diseases such as Alzheimer and developmental disorders caused by defects in the Notch pathway

30. A method for the production of a pharmaceutical composition comprising carrying out the method of any of No. 22 to 27 to identify a molecule that modulates the function, activity, composition or formation of said complex, and further comprising mixing the identified molecule with a pharmaceutically acceptable carrier.

31. A method for diagnosing or screening for the presence of a disease or disorder or a predisposition for developing a disease or disorder in a subject, which disease or disorder is characterized by an aberrant amount of, activity of, component composition of, or intracellular localization of the complex of any one of No. 1 to 8, comprising determining the amount of, activity of, protein components of, and/or intracellular localization of, said complex and/or the transcription level of a gene dependent on the complex and/or the abundance and/or activity of a protein or protein complex dependent on the function of the complex and/or product of a gene dependent on the complex in a comparative sample derived from a subject, wherein a difference in said amount, activity, or protein components of, said complex in an analogous sample from a subject not having the disease or disorder or predisposition indicates the presence in the subject of the disease or disorder or predisposition in the subject.

32. The method of No. 31, wherein the amount of said complex is determined.

33. The method of No. 31, wherein the activity of said complex is determined.

34. The method of No. 33, wherein said determining step comprises isolating from the cell or organism said complex to produce said isolated complex and contacting said isolated complex in the presence or absence of a candidate molecule with a physiological substrate of any of the complexes according to any of claims 1 to 4 and determine whether said substrate is processed.

35. The method of No. 31, wherein the amount of the individual protein components of said complex are determined.

36. The method of No. 35, wherein said determining step comprises determining whether
(i) Sambiasin-1 (SEQ ID No: 1) or a functionally active derivative thereof, or a functionally active fragment thereof, or a homolog thereof, or a variant of Sambiasin-1 encoded by a nucleic acid that hybridizes to the Sambiasin-1 nucleic acid or its complement under low stringency conditions, and/or
(ii) Presenilin-1 (SEQ ID No: 2), or a functionally active derivative thereof, or a functionally active fragment thereof, or a homolog thereof, or a variant of Presenilin-1 encoded by a nucleic acid that hybridizes to the Presenilin-1 nucleic acid or its complement under low stringency conditions, and/or
(iii) Nicastrin (SEQ ID No: 3), or a functionally active derivative thereof, or a functionally active fragment thereof, or a homolog thereof, or a variant of Nicastrin encoded by a nucleic acid that hybridizes to the Nicastrin nucleic acid or its complement under low stringency conditions, are present in the complex and wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.

37. The complex of any one of No. 1,2,3,4,5,6,7, 8, or proteins of any of No. 9, 10 or the antibody or fragment of No. 14, for use in a method of diagnosing a disease or disorder such as neurodegenerative diseases such as Alzheimer and developmental disorders caused by defects in the Notch pathway.

38. A method for treating or preventing a disease or disorder characterized by an aberrant amount of, activity of, component composition of or intracellular localization of, the complex of anyone of No. 1,2,3,4,5,6,7,8 comprising administering to a subject in need of such treatment or prevention a therapeutically effective amount of one or more molecules that modulate the amount of, activity of, or protein components of, said complex.

39. The method according to No. 38, wherein said disease or disorder involves decreased levels of the amount or activity of said complex.

40. The method according to No. 39, wherein said disease or disorder involves i increased levels of the amount or activity of said complex.

41. Complex of any of No. 1,2,3,4,5,6,7,8 and/or protein selected from the following proteins
(i) Sambiasin-1 (SEQ ID No: 1) or a functionally active derivative thereof, or a functionally active fragment thereof, or a homolog thereof, or a variant of Sambiasin-1 encoded by a nucleic acid that hybridizes to the Sambiasin-1 nucleic acid or its complement under low stringency conditions, or
(ii) Presenilin-1 (SEQ ID No: 2), or a functionally active derivative thereof, or a functionally active fragment thereof, or a homolog thereof, or a variant of Presenilin-1 encoded by a nucleic acid that hybridizes to the Presenilin-1 nucleic acid or its complement under low stringency conditions, or
(iii) Nicastrin (SEQ ID No: 3), or a functionally active derivative thereof, or a functionally active fragment thereof, or a homolog thereof, or a variant of Nicastrin encoded by a nucleic acid that hybridizes to the Nicastrin nucleic acid or its complement under low stringency conditions, wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.,
   as a target for an active agent of a pharmaceutical, preferably a drug target in the treatment or prevention of a disease or disorder such as neurodegenerative diseases such as Alzheimer and developmental disorders caused by defects in the Notch pathway.

### b 3.1 Definitions

The term “activity” as used herein, refers to the function of a molecule in its broadest sense. It generally includes, but is not limited to, biological, biochemical, physical or chemical functions of the molecule. It includes for example the enzymatic activity, the ability to interact with other molecules and ability to activate, facilitate, inhibit, suppress or destabilize the function of other molecules, stability, ability to localize to certain subcellular locations.

The term “agonist” as used herein, means a molecule which modulates the formation of a protein complex as provided herein or which, when bound to a complex or protein of the inventionor a molecule in the protein complex, increases the amount of, or prolongs the duration of, the activity of the complex. The stimulation may be direct or indirect, including effects on the expression of a gene encoding a member of the protein complex, or by a competitive or non-competitive mechanism. Agonists may include proteins, nucleic acids, carbohydrates or any other organic or anorganic molecule or metals. Agonists also include a functional peptide or peptide fragment derived from a protein member of the complexes of the invention or a protein member itself of the complexes of the invention. Preferred activators are those which, when added to the complex and/or the protein of the invention under physiological conditions and/or in vitro assays, including diagnostic or prognostic assays, result in a change of the level of any of the activities of the protein complex and/or the proteins of the invention as exemplary illustrated above by at least 10%, at least 25%, at least 50%, at least 100%, at least, 200%, at least 500% or at least 1000% at a concentration of the activator 1 μg ml⁻¹, 10 μg ml⁻¹, 100 μg ml⁻¹, 500 μg ml⁻¹, 1 mg ml⁻¹, 10 mg ml⁻¹ or 100 mg ml⁻¹. Any combination of the above mentioned degrees of percentages and concentration may be used to define anagonist of the invention, with greater effect at lower concentrations being preferred.

The term “amount” as used herein and as applicable to the embodiment described relates to the amount of the particular protein or protein complex described, including the value of null, i.e. where no protein or protein complex described in that particular embodiment is present under the or any of the conditions which might be specified in that particular embodiment.

The term “animal” as used herein includes, but is not limited to mammals, preferably mammals such as cows, pigs, horses, mice, rats, cats, dogs, sheeps, goats and most preferably humans. Other animals used in agriculture, such as chickens, ducks etc are also included in the definition as used herein.

The term “animal” as used herein does not include humans if being used in the context of genetic alterations to the germline

The term “antagonist” as used herein, means a molecule which modulates the formation of a protein complex or which, when bound to a complex or protein of the inventionor a molecule in the protein complex, decreases the amount of, or the duration or level of activity of the complex. The effect may be direct or indirect, including effects on the expression of a gene encoding a member of the protein complex, or by a competitive or non-competitive mechanism. Antagonists may include proteins, including antibodies, nucleic acids, carbohydrates or any other organic or anorganic molecule or metals. Antagonists also include a functional peptide or peptide fragment derived from a protein member of the complexes of the invention or a protein member itself of the complexes of the invention. Preferred antagonists are those which, when added to the complex and/or the protein of the invention under physiological conditions and/or in vitro assays, including diagnostic or prognostic assays, result in a change of the level of any of the activities of the protein complex and/or the proteins of the invention as exemplary illustrated above by at least 10%, at least 20%, at least 30%, at least 40% at least 50%, at least 60%, at least 70%, at least 80%, at least 90%, at least 95% or at least 99% at a concentration of the inhibitor of 1 μg ml⁻¹, 10 μg ml⁻¹, 100 μg ml⁻¹, 500 μg ml^{31 1}, 1 mg ml⁻¹, 10 mg ml⁻¹ or 100 mg ml⁻¹.

Any combination of the above mentioned degrees of percentages and concentration may be used to define antaagonist of the invention, with greater effect at lower concentrations being preferred.

The term “antibodies” as used herein, include include, but are not limited to, polyclonal, monoclonal, chimeric, single chain, Fab fragments, and an Fab expression library.

The term “association” as used herein and as applicable to the embodiment described is being used interchangeably with the term “protein complex”.

The term “amount” as used herein and as applicable to the embodiment described relates to the amount of the particular protein or protein complex described, including the value of null, i.e. where no protein or protein complex described in that particular embodiment is present under the or any of the conditions which might be specified in that particular embodiment.

The term “binding” as used herein means a stable or transient association between two molecules, including electrostatic, hydrophobic, ionic and/or hydrogen-bond interaction under physiological conditions and/or conditions being used in diagnostic or prognostic method or process or procedure.

The term “carrier” as used herein refers to a diluent, adjuvant, excipient, or vehicle with which the therapeutic is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water is a preferred carrier when the pharmaceutical composition is administered orally. Saline and aqueous dextrose are preferred carriers when the pharmaceutical composition is administered intravenously. Saline solutions and aqueous dextrose and glycerol solutions are preferably employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the Therapeutic, preferably in purified form, together with a suitable amount of carrier so as to provide the form for proper administration to the patient. The formulation should suit the mode of administration.

If not stated otherwise, the terms “complex” and “protein complex” or “Presenilin-complex” are used interchangeably herein and refer to a complex of proteins as provided herein that is able to perform one or more functions of the wild type protein complex. The protein complex may or may not include and/or be associated with other molecules such as nucleic acid, such as RNA or DNA, or lipids or further cofactors or moieties selected from a metal ions,hormones, second messengers, phosphate, sugars. A “complex” of the invention may also be part of or a unit of a larger physiological protein assembly.

If not stated otherwise, the term “compound” as used herein are include but are not limited to peptides, nucleic acids, carbohydrates, natural product extract librariesorganic molecules, preferentially small organic molecules, anorganic molecules, including but not limited to chemicals, metals and organometallic molecules

The terms “derivatives” or “analogs of component proteins” or “variants” as used herein include, but are not limited, to molecules comprising regions that are substantially homologous to the component proteins, in various embodiments, by at least 30%, 40%, 50%, 60%, 70%, 80%, 90%, 95% or 99% identity over an amino acid sequence of identical size or when compared to an aligned sequence in which the alignment is done by a computer homology program known in the art, or whose encoding nucleic acid is capable of hybridizing to a sequence encoding the component protein under stringent, moderately stringent, or nonstringent conditions. It means a protein which is the outcome of a modification of the naturally occurring protein, by amino acid substitutions, deletions and additios, respectively, which derivatives still exhibit the biological function of the naturally occurring protein although not necessarily to the same degree. The biological function of such proteins can e.g. be examined by suitable available in vitro assays as provided in the invention.

The term “gamma-secretase activity” as used herein refers to the biochemical activity which has been linked to the pathological cleavage of the Alzheimer Precursor Protein (APP). (see. f.e.: Nunan H and Small D H (2000) FEBS Lett 483: 6-10; Selkoe D J (1994) Annu Rev Cell Biol 10: 373-4902; Esler W P and Wolfe M S (2001) Science 293: 1449-54.

The term “gene” as used herein refers to a nucleic acid comprising an open reading frame encoding a polypeptide of, if not stated otherwise, the present invention, including both exon and optionally intron sequences.

The term “homolog” or “homologous gene products” as used herein means a protein in another species, preferably mammals, which performs the same biological function as the a protein component of the complex further described herein. Such homologs are also termed “orthologue gene products”. The algorithm for the detection of orthologue gene pairs from humanst and mammalians or other species uses the whole genome of these organisms. First, pairwise best hits are retrieved, using a full Smith-Waterman alignment of predicted proteins. To further improve reliability, these pairs are clustered with pairwise best hits involving Drosophila melanogaster and C. elegans proteins. Such analysis is given, e.g., in Nature, 2001, 409:860-921. The homologs of the proteins according to the invention can either be isolated based on the sequence homology of the genes encoding the proteins provided herein to the genes of other species by cloning the respective gene applying conventional technology and expressing the protein from such gene, or by isolating proteins of the other species by isolating the analogous complex according to the methods provided herein or to other suitable methods commonly known in the art.

The term “host cells” or, were applicable, “cells” or “hosts” as used herein is intended to be understood in a broadest sense and include, but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used. It is understood that this term not only refers to the particular subject cell but to the progeny or potetial progeny of such a cell. Because certain modifications may occur in succeeding generations due to either mutation of environmental inlcuences, such progeny may not, in fact, be identical to the parent cell, but are still included within the scope of the term as used herein.

The term “Nicastrin” as used herein refers to a class of proteins which has been also been known to associate with presenilin (s. f.e. patent application WO-01/67109).

The term “nuleic acid” as used herein refers to polynucleotides such as deoxyribonucleic acid (DNA), and, where appropriate, ribonucleic acid (RNA). They may also be polynucleotides which include within them synthetic or modified nucleotides. A number of different types of modification to polynucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Such modifications may be carried out in order to enhance the in vivo activity or lifespan of polynucleotides of the invention. Polynucleotides according to the invention may be produced recombinantly, synthetically, or by any means available to those of skill in the art. They may also be cloned by standard techniques. The polynucleotides are typically provided in isolated and/or purified form. As applicable to the embodiment being described, they include both single stranded and double-stranded polynucleotides

The term “percent identity”, as used herein, means the number of identical residues as defined by an optimal alignment using the Smith-Waterman algorithm divided by the length of the overlap multiplied by 100. The alignment is performed by the search program (W. R. Pearson, 1991, Genomics 11:635-650) with the constraint to align the maximum of both sequences.

The terms “polypeptides” and “proteins” are, where applicable, used interchangeably herein. They may be chemically modified, e.g. post-translationally modified. For example, they may be glycosylated or comprise modified amino acid residues. They may also be modified by the addition of a signal sequence to promote their secretion from a cell where the polypeptide does not naturally contain such a sequence. They maybe tagged with a tag. They may be tagged with different labels which may assists in identification of the proteins in a protein complex. Polypeptides/proteins for use in the invention may be in a substantially isolated form. It will be understood that the polypeptid/protine may be mixed with carriers or diluents which will not interfere with the intended purpose of the polypeptide and still be regarded as substantially isolated. A polypeptide/protein for use in the invention may also be in a substantially purified form, in which case it will generally comprise the polypeptide in a preparation in which more than 50%, e.g. more than 80%, 90%, 95% or 99%, by weight of the polypeptide in the preparation is a polypeptide of the invention.

The term “presenilin” as used herein refer to a family of transmembrane proteins (see f.e.: (see. fe.: Nunan H and Small D H (2000) FEBS Lett 483: 6-10; Selkoe D J (1994) Annu Rev Cell Biol 10: 373-4902; Esler W P and Wolfe M S (2001) Science 293: 1449-54.

“Target for therapeutic drug” means that the respective protein (target) can bind the active ingredient of a pharmaceutical composition and thereby changes its biological activity in response to the drug binding.

The term “tag” as used herein is meant to be understood in its broadest sense and to include, but is not limited to any suitable enzymatic, fluorescent, or radioactive labels and suitable epitopes, incuding but not limited to HA-tag, Myc-tag, T7, His-tag, FLAG-tag, Calmodulin binding proteins, glutatione-S-transferase, strep-tag, KT3-epitope, EEF-epitpopes, green-fluorescent protein and variants thereof.

The term “Therapeutics” as used herein, includes, but are not limited to, a protein complex of the present invention, the individual component proteins, and analogs and derivatives (including fragments); antibodies thereto; nucleic acids encoding the component protein, and analogs or derivatives, thereof); component protein antisense nucleic acids, and agents that modulate complex formation and/or activity (i.e., agonists and antagonists).

The term “vector” as used herein means a nucleic acid molecule capable of transporting another nucleic acid sequence to which it has been linked. Preferred vectors are those capable of autonomous replication and/or expression of nueclic acids to which they linked. The terms “plasmid” and “vector” are used interchangeably herein when applicable to the embodiment. However, vectors other than plasmids are also included herein. The expression elements of vectors vary in their strengths and specificities. Depending on the host-vector system utilized, any one of a number of suitable transcription and translation elements may be used.

### 4. DETAILED DESCRIPTION OF THE INVENTION

Overview:

An object of the present invention was to identify novel interactors of Presenilin to elucidate the molecular basis for the biochemical processes associated with Presenilin and thus to provide a better understanding in order to develop new therapeutic approaches and to provide new drug targets for the treatment of neurodegenerative diseases such as Alzheimer's disease and disorders caused by defects in the Notch-pathway.

Said objects have been achieved by the findings described below:

By applying the process as described below (EXAMPLES), the present invention provides a direct action between a presenilin and a proteins herein call Sambiasins. With the previous finding of a direct action between Presenilin and Nicastrin as described in WO-01/67109, the invention thus also provides a protein complex comprising Sambiasin, Presenilin and Nicastrin.

The interactions provided herein provides a new therapeutic intervention point in disorders involving defective presenilin function, such as neurodegenerative diseases and more specifically Alzheimers disease or diseases caused by defects in the Notch-pathway. In addition Sambiasin is now proposed as a target for agents which may be useful in the treatment of neurodegenerative diseases such as Alzheimers disease or disorders caused by defects in the Notch-pathway and as a tool for the identification of such agents.

Furthermore, the invention provides a novel form of Sambiasin.

From the amino acid sequence a transmembrane protein with seven membrane spanning alpha-helices is predicted. Functional prediction programmes predict a non-enzymatic protein involved in transport and binding. The membrane spanning helices do not show any amphipathic character. There is no match to any known structure.

The invention thus relates to the following embodiments:

1. A protein complex comprising
(a) a first protein, or a functionally active fragment or functionally active derivative thereof, which first protein is selected from the group consisting of:
   (i) Sambiasin-1 (SEQ ID No: 1) or a functionally active derivative thereof, or a functionally active fragment thereof, or a homolog thereof, or a variant of Sambiasin-1 encoded by a nucleic acid that hybridizes to the Sambiasin-1 nucleic acid or its complement under low stringency conditions,
(b) a second protein, or a functionally active fragment or functionally active derivative thereof, which second protein is selected from the group consisting of:
   (i) Presenilin-1 (SEQ ID No: 2), or a functionally active derivative thereof, or a functionally active fragment thereof, or a homolog thereof, or a variant of Presenilin-1 encoded by a nucleic acid that hybridizes to the Presenilin-1 nucleic acid or its complement under low stringency conditions,
   (ii) Nicastrin (SEQ ID No: 3), or a functionally active derivative thereof, or a functionally active fragment thereof, or a homolog thereof, or a variant of Nicastrin encoded by a nucleic acid that hybridizes to the Nicastrin nucleic acid or its complement under low stringency conditions, wherein said first protein and said second protein are members of a native cellular complex, and wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.

2. A protein complex comprising Sambiasin-1 (SEQ ID No: 1) or Sambiasin-2 (SEQ ID No: 4) and Presenilin1 (SEQ ID No: 2) or Presenilin-2 (SEQ ID No: 5).

3. A protein complex according to No. 1,2 further comprising Nicastrin (SEQ ID No: 3)

4. A protein complex comprising Sambiasin-1 SEQ ID No: 1) and Presenilin-1 (SEQ ID No: 2) and Nicastrin (SEQ ID No: 3).

5. The complex of No. 1,2,3,4 comprising a functionally active derivative of any of the proteins of said complex, wherein the functionally active derivative is a fusion protein comprising said protein fused to an amino acid sequence different from said protein.

6. The complex of claim 5 wherein the functionally active derivative is a fusion protein comprising said protein fused to an affinity tag or label.

7. The complex of No. 1,2,3,4 comprising a fragment of any of the proteins of said complex, which fragment binds to another protein component of said complex.

8. The complex of No. 1,2,3,4,5,6,7 that is involved in the gamma-secretase activity.

9. Protein comprising the amino acid sequence of SEQ ID No: 1, or a functionally active derivative thereof, or a functionally active fragment thereof, or a homolog thereof, or a variant of Sambiasin-1 encoded by a nucleic acid that hybridizes to the Sambiasin-1 nucleic acid or its complement under low stringency conditions, wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C., with the provisio that the protein does not have the amino acid sequence according to SEQ ID 6.

10. Protein comprising the amino acid sequence of SEQ ID No: 1.

11. Nucleic acid encoding a protein according to No 9 or 10.

12. Construct, preferably a vector construct, comprising
(a) a nucleic acid according to No. 11 and at least one further nucleic acid which is normally not associated with said nucleic acid, or
(b) at least two separate nucleic acid sequences each encoding a different protein of any of the proteins, or a functionally active fragment or a functionally active derivative thereof according to No. 1.

13. Host cell, containing a vector comprising at least one of the nucleic acid of No. 11 and/or any of the constructs of No. 12 or containing several vectors each comprising at least the nucleic acid sequence encoding at least one of the proteins, or functionally active fragments or functionally active derivatives thereof selected from the first group of proteins according to No. 1.

14. An antibody or a fragment of said antibody containing the binding domain thereof, which binds the complex of any No. 1 to 8 and which does not bind the first protein when uncomplexed or the second protein when uncomplexed and/or an antibody or a fragment of said antibody containing the binding domain thereof which binds to any of the group of proteins according to any of No. 9, 10.

15. A kit comprising in one or more container the complex of any of No. 1 to 8 and/or the proteins of any of No. 9, 10, optionally together with an antibody according to No. 14 and/or further components such as reagents and working instructions.

16. A kit according to No. 15 for the diagnosis or prognosis of a disease or a disease risk, preferentially for a disease or disorder such as neurodegenerative diseases such as Alzheimer and developmental disorders caused by defects in the Notch pathway.

17. Array, in which at least a complex according to any of No. 1 to 8 and/or any of the proteins of any of No. 9 or 10 and/or at least one antibody according to No. 14 is attached to a solid carrier.

18. A process for processing the physiological substrates of any of the complexes of any of No. 1,2,3,4 comprising the step of bringing into contact a complex of any of No. 1 to 7 with said substrate, such that said substrate is processed.

19. A pharmaceutical composition comprising the protein complex of No. 1,2,3,4,5,6,7 or 8 and a pharmaceutically acceptable carrier and/or any of the proteins of No. 9 or 10 and a pharmaceutically acceptable carrier.

20. A pharmaceutical composition according to No. 19 for the treatment of diseases and disorders such as neurodegenerative diseases, such as Alzheimer, and/or developmental disorders caused by defects in the Notch pathway.

21. A method for screening for a molecule that binds to the complex of anyone of No. 1 to 8 and/or any of the proteins of No. 9 or 10, comprising the following steps:
(a) exposing said complex or protein, or a cell or organism containing same, to one or more candidate molecules; and
(b) determining whether said candidate molecule is bound to the complex or protein.

b 22. A method for screening for a molecule that modulates directly or indirectly the function, activity, composition or formation of the complex of any one of No. 1 to 8 comprising the steps of:
(a) exposing said complex, or a cell or organism containing said complex to one or more candidate molecules; and
(b) determining the amount of, activity of, protein components of, and/or intracellular localization of, said complex and/or the transcription level of a gene dependend on the complex and/or the abundance and/or activity of a protein or protein complex dependend on the function of the complex and/or product of a gene dependent on the complex in the presence of the one or more candidate molecules, wherein a change in said amount, activity, protein components or intracellular localization relative to said amount, activity, protein components and/or intracellular localization and/or a change in the transcription level of a gene dependend on the complex and/or the abundance and/or activity of a protein or protein complex dependent on the function of the complex and/or product of a gene dependent on the complex in the absence of said candidate molecules indicates that the molecule modulates function, activity or composition of said complex.

23. The method of No. 22 wherein the amount of said complex is determined.

24. The method of No. 22, wherein the activity of said complex is determined.

25. The method of No. 24, wherein said determining step comprises isolating from the cell or organism said complex to produce said isolated complex and contacting said isolated complex in the presence or absence of a candidate molecule with a physiological substrate of any of the complexes according to No. 1,2,3,4 and determine whether said substrate is processed.

26. The method of No. 22, wherein the amount of the individual protein components of said complex are determined.

27. The method of No. 26, wherein said determining step comprises determining whether
(i) Sambiasin-1 (SEQ ID No: 1), or a functionally active derivative thereof, or a functionally active fragment thereof, or a homolog thereof, or a variant of Sambiasin-1 encoded by a nucleic acid that hybridizes to the Sambiasin-1 nucleic acid or its complement under low stringency conditions, and/or
(ii) Presenilin-1 (SEQ ID No: 2), or a functionally active derivative thereof, or a functionally active fragment thereof, or a homolog thereof, or a variant of Presenilin-1 encoded by a nucleic acid that hybridizes to the Presenilin-1 nucleic acid or its complement under low stringency conditions, and/or
(iii) Nicastrin (SEQ ID No: 3), or a functionally active derivative thereof, or a functionally active fragment thereof, or a homolog thereof, or a variant of Nicastrin encoded by a nucleic acid that hybridizes to the Nicastrin nucleic acid or its complement under low stringency conditions,
   are present in the complex and wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.

28. The method of any of No. 22 to 27, wherein said method is a method of screening for a drug for treatment or prevention of a disease or disorder such as neurodegenerative diseases such as Alzheimer and developmental disorders caused by defects in the Notch pathway.

29. Use of a molecule that modulates the amount of, activity of, or the protein components of the complex of any one of No. 1 to 8 for the manufacture of a medicament for the treatment or prevention of a disease or disorder such as neurodegenerative diseases such as Alzheimer and developmental disorders caused by defects in the Notch pathway

30. A method for the production of a pharmaceutical composition comprising carrying out the method of any of No. 22 to 27 to identify a molecule that modulates the function, activity, composition or formation of said complex, and further comprising mixing the identified molecule with a pharmaceutically acceptable carrier.

31. A method for diagnosing or screening for the presence of a disease or disorder or a predisposition for developing a disease or disorder in a subject, which disease or disorder is characterized by an aberrant amount of, activity of, component composition of, or intracellular localization of the complex of any one of No. 1 to 8, comprising determining the amount of, activity of, protein components of, and/or intracellular localization of, said complex and/or the transcription level of a gene dependent on the complex and/or the abundance and/or activity of a protein or protein complex dependent on the function of the complex and/or product of a gene dependent on the complex in a comparative sample derived from a subject, wherein a difference in said amount, activity, or protein components of, said complex in an analogous sample from a subject not having the disease or disorder or predisposition indicates the presence in the subject of the disease or disorder or predisposition in the subject.

32. The method of No. 31, wherein the amount of said complex is determined.

33. The method of No. 31, wherein the activity of said complex is determined.

34. The method of No. 33, wherein said determining step comprises isolating from the cell or organism said complex to produce said isolated complex and contacting said isolated complex in the presence or absence of a candidate molecule with a physiological substrate of any of the complexes according to any of claims 1 to 4 and determine whether said substrate is processed.

35. The method of No. 31, wherein the amount of the individual protein components of said complex are determined.

36. The method of No. 35, wherein said determining step comprises determining whether
(i) Sambiasin-1 (SEQ ID No: 1) or a functionally active derivative thereof, or a functionally active fragment thereof, or a homolog thereof, or a variant of Sambiasin-1 encoded by a nucleic acid that hybridizes to the Sambiasin-1 nucleic acid or its complement under low stringency conditions, and/or
(ii) Presenilin-1 (SEQ ID No: 2), or a functionally active derivative thereof, or a functionally active fragment thereof, or a homolog thereof, or a variant of Presenilin-1 encoded by a nucleic acid that hybridizes to the Presenilin-1 nucleic acid or its complement under low stringency conditions, and/or
(iii) Nicastrin (SEQ ID No: 3), or a functionally active derivative thereof, or a functionally active fragment thereof, or a homolog thereof, or a variant of Nicastrin encoded by a nucleic acid that hybridizes to the Nicastrin nucleic acid or its complement under low stringency conditions,
   are present in the complex and wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.

37. The complex of any one of No. 1,2,3,4,5,6,7, 8, or proteins of any of No. 9, 10 or the antibody or fragment of No. 14, for use in a method of diagnosing a disease or disorder such as neurodegenerative diseases such as Alzheimer and developmental disorders caused by defects in the Notch pathway.

38. A method for treating or preventing a disease or disorder characterized by an aberrant amount of, activity of, component composition of or intracellular localization of, the complex of anyone of No. 1,2,3,4,5,6,7,8 comprising administering to a subject in need of such treatment or prevention a therapeutically effective amount of one or more molecules that modulate the amount of, activity of, or protein components of, said complex.

39. The method according to No. 38, wherein said disease or disorder involves decreased levels of the amount or activity of said complex.

40. The method according to No. 39, wherein said disease or disorder involves i increased levels of the amount or activity of said complex.

41. Complex of any of No. 1,2,3,4,5,6,7,8 and/or protein selected from the following proteins
(i) Sambiasin-1 (SEQ ID No: 1) or a functionally active derivative thereof, or a functionally active fragment thereof, or a homolog thereof, or a variant of Sambiasin-1 encoded by a nucleic acid that hybridizes to the Sambiasin-1 nucleic acid or its complement under low stringency conditions, or
(ii) Presenilin-1 (SEQ ID No: 2), or a functionally active derivative thereof, or a functionally active fragment thereof, or a homolog thereof, or a variant of Presenilin-1 encoded by a nucleic acid that hybridizes to the Presenilin-1 nucleic acid or its complement under low stringency conditions, or
(iii) Nicastrin (SEQ ID No: 3), or a functionally active derivative thereof, or a functionally active fragment thereof, or a homolog thereof, or a variant of Nicastrin encoded by a nucleic acid that hybridizes to the Nicastrin nucleic acid or its complement under low stringency conditions, wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.,
   as a target for an active agent of a pharmaceutical, preferably a drug target in the treatment or prevention of a disease or disorder such as neurodegenerative diseases such as Alzheimer and developmental disorders caused by defects in the Notch pathway. Animal models are also provided herein Preferably, the protein components of the complexes described herein are all mammalian proteins. The complexes can also consist only of the respective homologs from other mammals such as mouse, rat, pig, cow, dog, monkey, sheep or horse or other species such as Drosophila, C.elegans or chicken. In another preferred embodiment, the complexes are a mixture of proteins from two or more species.

### 4.1. Protein Complexes/Proteins of the Invention

The protein complexes of the present invention and their component proteins are described above.

The protein complexes and component proteins can be obtained by methods well known in the art for protein purification and recombinant protein expression For example, the interaction partners (the protein complex) can be isolated by immunoprecipitation of the component proteins and combining the immunoprecipitated proteins. In addition, the protein complexes of the present invention can be isolated using the TAP method described in WO 00/09716 and Rigaut et al., 1999, Nature Biotechnology 17:1030-1032, which are each incorporated by reference in their entirety. The protein complexes can also be produced by recombinantly expressing the component proteins and combining the expressed proteins.

The amino acid sequences of the component proteins of the protein complexes of the present invention are provided herein (SEQ ID NOS:1-7), and can be obtained by any method known in the art, e.g., by PCR amplification using synthetic primers hybridizable to the 3′ and 5′ ends of each sequence, and/or by cloning from a cDNA or genomic library using an oligonucleotide specific for each nucleotide sequence.

Homologs (e.g., nucleic acids encoding component proteins from other species) or other related sequences (e.g., variants, paralogs) which are members of a native cellular protein complex can be obtained by low, moderate or high stringency hybridization with all or a portion of the particular nucleic acid sequence as a probe, using methods well known in the art for nucleic acid hybridization and cloning.

Exemplary moderately stringent hybridization conditions are as follows: prehybridization of filters containing DNA is carried out for 8 hours to overnight at 65□ C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 hours at 65° C. in prehybridization mixture containing 100 μg/ml denatured salmon sperm DNA and 5-20×106 cpm of 32P-labeled probe. Washing of filters is done at 37° C. for 1 hour in a solution containing 2×SSC, 0.01% PVP, 0.01% Ficoll, and 0.01% BSA. This is followed by a wash in 0.1×SSC at 50° C. for 45 min before autoradiography. Alternatively, exemplary conditions of high stringency are as follows: e.g., hybridization to filter-bound DNA in 0.5 M NaHPO4, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. 1, Green Publishing Associates, Inc., and John Wiley & sons, Inc., New York, at p. 2.10.3). Other conditions of high stringency which may be used are well known in the art. Exemplary low stringency hybridization conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 μg/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.

For recombinant expression of one or more of the proteins, the nucleic acid containing all or a portion of the nucleotide sequence encoding the protein can be inserted into an appropriate expression vector, i.e., a vector that contains the necessary elements for the transcription and translation of the inserted protein coding sequence. The necessary transcriptional and translational signals can also be supplied by the native promoter of the component protein gene, and/or flanking regions.

A variety of host-vector systems may be utilized to express the protein coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

In a preferred embodiment, a complex of the present invention is obtained by expressing the entire coding sequences of the component proteins in the same cell, either under the control of the same promoter or separate promoters. In yet another embodiment, a derivative, fragment or homolog of a component protein is recombinantly expressed. Preferably the derivative, fragment or homolog of the protein forms a complex with the other components of the complex, and more preferably forms a complex that binds to an anti-complex antibody. Such an antibody is further described infra.

Any method available in the art can be used for the insertion of DNA fragments into a vector to construct expression vectors containing a chimeric gene consisting of appropriate transcriptional/translational control signals and protein coding sequences. These methods may include in vitro recombinant DNA and synthetic techniques and in vivo recombinant techniques (genetic recombination). Expression of nucleic acid sequences encoding a component protein, or a derivative, fragment or homolog thereof, may be regulated by a second nucleic acid sequence so that the gene or fragment thereof is expressed in a host transformed with the recombinant DNA molecule(s). For example, expression of the proteins may be controlled by any promoter/enhancer known in the art. In a specific embodiment, the promoter is not native to the gene for the component protein. Promoters that may be used can be selected from among the many known in the art, and are chosen so as to be operative in the selected host cell.

In a specific embodiment, a vector is used that comprises a promoter operably linked to nucleic acid sequences encoding a component protein, or a fragment, derivative or homolog thereof, one or more origins of replication, and optionally, one or more selectable markers (e.g., an antibiotic resistance gene).

In another specific embodiment, an expression vector containing the coding sequence, or a portion thereof, of a component protein, either together or separately, is made by subcloning the gene sequences into the EcoRI restriction site of each of the three pGEX vectors (glutathione S-transferase expression vectors; Smith and Johnson, 1988, Gene 7:3140). This allows for the expression of products in the correct reading frame.

Expression vectors containing the sequences of interest can be identified by three general approaches: (a) nucleic acid hybridization, (b) presence or absence of “marker” gene function, and (c) expression of the inserted sequences. In the first approach, coding sequences can be detected by nucleic acid hybridization to probes comprising sequences homologous and complementary to the inserted sequences. In the second approach, the recombinant vector/host system can be identified and selected based upon the presence or absence of certain “marker” functions (e.g., resistance to antibiotics, occlusion body formation in baculovirus, etc.) caused by insertion of the sequences of interest in the vector. For example, if a component protein gene, or portion thereof, is inserted within the marker gene sequence of the vector, recombinants containing the encoded protein or portion will be identified by the absence of the marker gene function (e.g., loss of beta-galactosidase activity). In the third approach, recombinant expression vectors can be identified by assaying for the component protein expressed by the recombinant vector. Such assays can be based, for example, on the physical or functional properties of the interacting species in in vitro assay systems, e.g., formation of a complex comprising the protein or binding to an anti-complex antibody.

Once recombinant component protein molecules are identified and the complexes or individual proteins isolated, several methods known in the art can be used to propagate them. Using a suitable host system and growth conditions, recombinant expression vectors can be propagated and amplified in quantity. As previously described, the expression vectors or derivatives which can be used include, but are not limited to, human or animal viruses such as vaccinia virus or adenovirus; insect viruses such as baculovirus, yeast vectors; bacteriophage vectors such as lambda phage; and plasmid and cosmid vectors.

In addition, a host cell strain may be chosen that modulates the expression of the inserted sequences, or modifies or processes the expressed proteins in the specific fashion desired, Expression from certain promoters can be elevated in the presence of certain inducers; thus expression of the genetically-engineered component proteins may be controlled. Furthermore, different host cells have characteristic and specific mechanisms for the translational and post-translational processing and modification (e.g., glycosylation, phosphorylation, etc.) of proteins. Appropriate cell lines or host systems can be chosen to ensure that the desired modification and processing of the foreign protein is achieved. For example, expression in a bacterial system can be used to produce an unglycosylated core protein, while expression in mammalian cells ensures “native” glycosylation of a heterologous protein. Furthermore, different vector/host expression systems may effect processing reactions to different extents.

In other specific embodiments, a component protein or a fragment, homolog or derivative thereof, may be expressed as fusion or chimeric protein product comprising the protein, fragment, homolog, or derivative joined via a peptide bond to a heterologous protein sequence of a different protein. Such chimeric products can be made by ligating the appropriate nucleic acid sequences encoding the desired amino acids to each other by methods known in the art, in the proper coding frame, and expressing the chimeric products in a suitable host by methods commonly known in the art. Alternatively, such a chimeric product can be made by protein synthetic techniques, e.g., by use of a peptide synthesizer. Chimeric genes comprising a portion of a component protein fused to any heterologous protein-encoding sequences may be constructed.

In particular, protein component derivatives can be made by altering their sequences by substitutions, additions or deletions that provide for functionally equivalent molecules. Due to the degeneracy of nucleotide coding sequences, other DNA sequences that encode substantially the same amino acid sequence as a component gene or cDNA can be used in the practice of the present invention. These include but are not limited to nucleotide sequences comprising all or portions of the component protein gene that are altered by the substitution of different codons that encode a functionally equivalent amino acid residue within the sequence, thus producing a silent change. Likewise, the derivatives of the invention include, but are not limited to, those containing, as a primary amino acid sequence, all or part of the amino acid sequence of a component protein, including altered sequences in which functionally equivalent amino acid residues are substituted for residues within the sequence resulting in a silent change. For example, one or more amino acid residues within the sequence can be substituted by another amino acid of a similar polarity that acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

In a specific embodiment, up to 1%, 2%, 5%, 10%, 15% or 20% of the total number of amino acids in the wild type protein are substituted or deleted; or 1, 2, 3, 4, 5, or 6 or up to 10 or up to 20 amino acids are inserted, substituted or deleted relative to the wild type protein.

In a specific embodiment of the invention, the nucleic acids encoding a protein component and protein components consisting of or comprising a fragment of or consisting of at least 6 (continuous) amino acids of the protein are provided. In other embodiments, the fragment consists of at least 10, 20, 30, 40, or 50 amino acids of the component protein. In specific embodiments, such fragments are not larger than 35, 100 or 200 amino acids. Derivatives or analogs of component proteins include, but are not limited, to molecules comprising regions that are substantially homologous to the component proteins, in various embodiments, by at least 30%, 40%, 50%,60%, 70%, 80%, 90%, 95% or 99% identity over an amino acid sequence of identical size or when compared to an aligned sequence in which the alignment is done by a computer homology program known in the art, or whose encoding nucleic acid is capable of hybridizing to a sequence encoding the component protein under stringent, moderately stringent, or nonstringent conditions.

In a specific embodiment, proteins are provided herein, which share an identical region of 20, 30, 40, 50 or 60 contiguos amino acids of the proteins provided herein (SEQ 1-7)

The protein component derivatives and analogs of the invention can be produced by various methods known in the art. The manipulations which result in their production can occur at the gene or protein level. For example, the cloned gene sequences can be modified by any of numerous strategies known in the art (Sambrook et al., 1989, Molecular Cloning, A Laboratory Manual, 2d Ed., Cold Spring Harbor Laboratory Press, Cold Spring Harbor, N.Y.). The sequences can be cleaved at appropriate sites with restriction endonuclease(s), followed by further enzymatic modification if desired, isolated, and ligated in vitro. In the production of the gene encoding a derivative, homolog or analog of a component protein, care should be taken to ensure that the modified gene retains the original translational reading frame, uninterrupted by translational stop signals, in the gene region where the desired activity is encoded.

Additionally, the encoding nucleic acid sequence can be mutated in vitro or in vivo, to create and/or destroy translation, initiation, and/or termination sequences, or to create variations in coding regions and/or form new restriction endonuclease sites or destroy pre-existing ones, to facilitate further in vitro modification. Any technique for mutagenesis known in the art can be used, including but not limited to, chemical mutagenesis and in vitro site-directed mutagenesis (Hutchinson et al., 1978, J. Biol. Chem 253:6551-6558), amplification with PCR primers containing a mutation, etc.

Once a recombinant cell expressing a component protein, or fragment or derivative thereof, is identified, the individual gene product or complex can be isolated and analyzed. This is achieved by assays based on the physical and/or functional properties of the protein or complex, including, but not limited to, radioactive labeling of the product followed by analysis by gel electrophoresis, immunoassay, cross-linking to marker-labeled product, etc.

The component proteins and complexes may be isolated and purified by standard methods known in the art (either from natural sources or recombinant host cells expressing the complexes or proteins), including but not restricted to column chromatography (e.g., ion exchange, affinity, gel exclusion, reversed-phase high pressure, fast protein liquid, etc.), differential centrifugation, differential solubility, or by any other standard technique used for the purification of proteins. Functional properties may be evaluated using any suitable assay known in the art.

Alternatively, once a component protein or its derivative, is identified, the amino acid sequence of the protein can be deduced from the nucleic acid sequence of the chimeric gene from which it was encoded. As a result, the protein or its derivative can be synthesized by standard chemical methods known in the art (e.g., Hunkapiller et al., 1984, Nature 310: 105-111).

Manipulations of component protein sequences may be made at the protein level. Included within the scope of the invention is a complex in which the component proteins or derivatives and analogs that are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to specific chemical cleavage by cyanogen bromide, trypsin, chymotrypsin, papain, V8 protease, NaBH₄, acetylation, formylation, oxidation, reduction, metabolic synthesis in the presence of tunicamycin, etc.

In specific embodiments, the amino acid sequences are modified to include a fluorescent label. In another specific embodiment, the protein sequences are modified to have a heterofunctional reagent; such heterofunctional reagents can be used to crosslink the members of the complex.

In addition, complexes of analogs and derivatives of component proteins can be chemically synthesized. For example, a peptide corresponding to a portion of a component protein, which comprises the desired domain or mediates the desired activity in vitro (e.g., complex formation) can be synthesized by use of a peptide synthesizer. Furthermore, if desired, non-classical amino acids or chemical amino acid analogs can be introduced as a substitution or addition into the protein sequence.

In cases where natural products are suspected of being mutant or are isolated from new species, the amino acid sequence of a component protein isolated from the natural source, as well as those expressed in vitro, or from synthesized expression vectors in vivo or in vitro, can be determined from analysis of the DNA sequence, or alternatively, by direct sequencing of the isolated protein. Such analysis can be performed by manual sequencing or through use of an automated amino acid sequenator.

The complexes can also be analyzed by hydrophilicity analysis (Hopp and Woods, 1981, Proc. Natl. Acad. Sci. USA 78:3824-3828). A hydrophilicity profile can be used to identify the hydrophobic and hydrophilic regions of the proteins, and help predict their orientation in designing substrates for experimental manipulation, such as in binding experiments, antibody synthesis, etc. Secondary structural analysis can also be done to identify regions of the component proteins, or their derivatives, that assume specific structures (Chou and Fasman, 1974, Biochemistry 13:222-23). Manipulation, translation, secondary structure prediction, hydrophilicity and hydrophobicity profile predictions, open reading frame prediction and plotting, and determination of sequence homologies, etc., can be accomplished using computer software programs available in the art.

Other methods of structural analysis including but not limited to X-ray crystallography (Engstrom, 1974 Biochem. Exp. Biol. 11:7-13), mass spectroscopy and gas chromatography (Methods in Protein Science, J. Wiley and Sons, New York, 1997), and computer modeling (Fletterick and Zoller, eds., 1986, Computer Graphics and Molecular Modeling, In: Current Communications in Molecular Biology, Cold Spring Harbor Laboratory, Cold Spring Harbor Press, New York) can also be employed.

### 4.2. Antibodies to Protein Complexes/Proteins of the Invention

According to the present invention, a protein complex of the present invention comprising Presenilin and CGI78 and optionally Nicastrin or functionally active fragments or functionally active derivatives of said proteins can be used as an immunogen to generate antibodies which immunospecifically bind such immunogen.

Such antibodies include, but are not limited to, polyclonal, monoclonal, chimeric, single chain, Fab fragments, and an Fab expression library. In a specific embodiment, antibodies to a complex comprising human protein components are produced. In another embodiment, a complex formed from a fragment of said first protein and a fragment of said second protein, which fragments contain the protein domain that interacts with the other member of the complex, are used as an immunogen for antibody production. In a preferred embodiment, the antibody specific for the complex in that the antibody does not bind the individual protein components of the complex.

Polyclonal antibodies can be prepared as described above by immunizing a suitable subject with a polypeptide of the invention as an immunogen. Preferred polyclonal antibody compositions are ones that have been selected for antibodies directed against a polypeptide or polypeptides of the invention. Particularly preferred polyclonal antibody preparations are ones that contain only antibodies directed against a polypeptide or polypeptides of the invention. Particularly preferred immunogen compositions are those that contain no other human proteins such as, for example, immunogen compositions made using a non-human host cell for recombinant expression of a polypeptide of the invention. In such a manner, the only human epitope or epitopes recognized by the resulting antibody compositions raised against this immunogen will be present as part of a polypeptide or polypeptides of the invention.

The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction. Alternatively, antibodies specific for a protein or polypeptide of the invention can be selected for (e.g., partially purified) or purified by, e.g., affinity chromatography. For example, a recombinantly expressed and purified (or partially purified) protein of the invention is produced as described herein, and covalently or non-covalently coupled to a solid support such as, for example, a chromatography column. The column can then be used to affinity purify antibodies specific for the proteins of the invention from a sample containing antibodies directed against a large number of different epitopes, thereby generating a substantially purified antibody composition, i.e., one that is substantially free of contaminating antibodies. By a substantially purified antibody composition is meant, in this context, that the antibody sample contains at most only 30% (by dry weight) of contaminating antibodies directed against epitopes other than those on the desired protein or polypeptide of the invention, and preferably at most 20%, yet more preferably at most 10%, and most preferably at most 5% (by dry weight) of the sample is contaminating antibodies. A purified antibody composition means that at least 99% of the antibodies in the composition are directed against the desired protein or polypeptide of the invention.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein, 1975, Nature 256:495-497, the human B cell hybridoma technique (Kozbor et al., 1983, Immunol. Today 4:72), the EBV-hybridoma technique (Cole et al., 1985, Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; and Shaw et al., 1988, J. Natl. Cancer Inst. 80:1553-1559); Morrison, 1985, Science 229:1202-1207; Oi et al., 1986, Bio/Techniques 4:214; U.S. Pat. No. 5,225,539; Jones et al., 1986, Nature 321:552-525; Verhoeyan et al., 1988, Science 239:1534; and Beidler et al., 1988, J. Immunol. 141:4053-4060.

Completely human antibodies are particularly desirable for therapeutic treatment of human patients. Such antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar, 1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope. (Jespers et al., 1994, Bio/technology 12:899-903).

Antibody fragments that contain the idiotypes of the complex can be generated by techniques known in the art. For example, such fragments include, but are not limited to, the F(ab′)2 fragment which can be produced by pepsin digestion of the antibody molecule; the Fab′ fragment that can be generated by reducing the disulfide bridges of the F(ab′)2 fragment; the Fab fragment that can be generated by treating the antibody molecular with papain and a reducing agent; and Fv fragments.

In the production of antibodies, screening for the desired antibody can be accomplished by techniques known in the art, e.g., ELISA (enzyme-linked immunosorbent assay). To select antibodies specific to a particular domain of the complex, or a derivative thereof, one may assay generated hybridomas for a product that binds to the fragment of the complex, or a derivative thereof, that contains such a domain. For selection of an antibody that specifically binds a complex of the present, or a derivative, or homolog thereof, but which does not specifically bind to the individual proteins of the complex, or a derivative, or homolog thereof, one can select on the basis of positive binding to the complex and a lack of binding to the individual protein components.

Antibodies specific to a domain of the complex, or a derivative, or homolog thereof, are also provided.

The foregoing antibodies can be used in methods known in the art relating to the localization and/or quantification of the complexes of the invention, e.g., for imaging these proteins, measuring levels thereof in appropriate physiological samples (by immunoassay), in diagnostic methods, etc. This hold true also for a derivative, or homolog thereof of a complex.

In another embodiment of the invention (see infra), an antibody to a complex or a fragment of such antibodies containing the antibody binding domain, is a Therapeutic.

### 4.3. Diagnostic, Prognostic, and Screening Uses of the Protein Complexes/Proteins of the Invention

The particular protein complexes and proteins of the present invention may be markers of normal physiological processes, and thus have diagnostic utility. Further, definition of particular groups of patients with elevations or deficiencies of a protein complex of the present invention, or wherein the protein complex has a change in protein component composition, can lead to new nosological classifications of diseases, furthering diagnostic ability. Examples for diseases or disorders in which the complexes provided herein are involved and/or associated with are neurodegenerative diseases such as Alzheimers disease and disorders caused by defects in the Notch-pathway.

Detecting levels of protein complexes, or individual component proteins that form the complexes, or detecting levels of the mRNAs encoding the components of the complex, may be used in diagnosis, prognosis, and/or staging to follow the course of a disease state, to follow a therapeutic response, etc.

A protein complex of the present invention and the individual components of the complex and a derivative, analog or subsequence thereof, encoding nucleic acids (and sequences complementary thereto), and anti-complex antibodies and antibodies directed against individual components that can form the complex, are useful in diagnostics. The foregoing molecules can be used in assays, such as immunoassays, to detect, prognose, diagnose, or monitor various conditions, diseases, and disorders characterized by aberrant levels of a complex or aberrant component composition of a complex, or monitor the treatment of such various conditions, diseases, and disorders.

In particular, such an immunoassay is carried out by a method comprising contacting a sample derived from a patient with an anti-complex antibody under conditions such that immunospecific binding can occur, and detecting or measuring the amount of any immunospecific binding by the antibody. In a specific aspect, such binding of antibody, in tissue sections, can be used to detect aberrant complex localization, or aberrant (e.g., high, low or absent) levels of a protein complex or complexes. In a specific embodiment, an antibody to the complex can be used to assay a patient tissue or serum sample for the presence of the complex, where an aberrant level of the complex is an indication of a diseased condition. By “aberrant levels” is meant increased or decreased levels relative to that present, or a standard level representing that present, in an analogous sample from a portion or fluid of the body, or from a subject not having the disorder.

The immunoassays which can be used include but are not limited to competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few known in the art.

Nucleic acids encoding the components of the protein complex and related nucleic acid sequences and subsequences, including complementary sequences, can be used in hybridization assays. The nucleic acid sequences, or subsequences thereof, comprising about at least 8 nucleotides, can be used as hybridization probes. Hybridization assays can be used to detect, prognose, diagnose, or monitor conditions, disorders, or disease states associated with aberrant levels of the mRNAs encoding the components of a complex as described, supra. In particular, such a hybridization assay is carried out by a method comprising contacting a sample containing nucleic acid with a nucleic acid probe capable of hybridizing to component protein coding DNA or RNA, under conditions such that hybridization can occur, and detecting or measuring any resulting hybridization.

In specific embodiments, diseases and disorders involving or characterized by aberrant levels of a protein complex or aberrant complex composition can be diagnosed, or its suspected presence can be screened for, or a predisposition to develop such disorders can be detected, by determining the component protein composition of the complex, or detecting aberrant levels of a member of the complex or un-complexed component proteins or encoding nucleic acids, or functional activity including, but not restricted to, binding to an interacting partner, or by detecting mutations in component protein RNA, DNA or protein (e.g., mutations such as translocations, truncations, changes in nucleotide or amino acid sequence relative to wild-type that cause increased or decreased expression or activity of a complex, and/or component protein.

By way of example, levels of a protein complex and the individual components of a complex can be detected by immunoassay, levels of component protein RNA or DNA can be detected by hybridization assays (e.g., Northern blots, dot blots, RNase protection assays), and binding of component proteins to each other (e.g., complex formation) can be measured by binding assays commonly known in the art. Translocations and point mutations in component protein genes can be detected by Southern blotting, RFLP analysis, PCR using primers that preferably generate a fragment spanning at least most of the gene by sequencing of genomic DNA or cDNA obtained from the patient, etc.

Assays well known in the art (e.g., assays described above such as immunoassays, nucleic acid hybridization assays, activity assays, etc.) can be used to determine whether one or more particular protein complexes are present at either increased or decreased levels, or are absent, in samples from patients suffering from a particular disease or disorder, or having a predisposition to develop such a disease or disorder, as compared to the levels in samples from subjects not having such a disease or disorder, or having a predisposition to develop such a disease or disorder. Additionally, these assays can be used to determine whether the ratio of the complex to the un-complexed components of the complex, is increased or decreased in samples from patients suffering from a particular disease or disorder, or having a predisposition to develop such a disease or disorder, as compared to the ratio in samples from subjects not having such a disease or disorder.

In the event that levels of one or more particular protein complexes (i.e., complexes formed from component protein derivatives, homologs, fragments, or analogs) are determined to be increased in patients suffering from a particular disease or disorder, or having a predisposition to develop such a disease or disorder, then the particular disease or disorder, or predisposition for a disease or disorder, can be diagnosed, have prognosis defined for, be screened for, or be monitored by detecting increased levels of the one or more protein complexes, increased levels of the mRNA that encodes one or more members of the one or more particular protein complexes, or by detecting increased complex functional activity.

Accordingly, in a specific embodiment of the present invention, diseases and disorders involving increased levels of one or more protein complexes can be diagnosed, or their suspected presence can be screened for, or a predisposition to develop such disorders can be detected, by detecting increased levels of the one or more protein complexes, the mRNA encoding both members of the complex, or complex functional activity, or by detecting mutations in the component proteins that stabilize or enhance complex formation, e.g., mutations such as translocations in nucleic acids, truncations in the gene or protein, changes in nucleotide or amino acid sequence relative to wild-type, that stabilize or enhance complex formation.

In the event that levels of one or more particular protein complexes are determined to be decreased in patients suffering from a particular disease or disorder, or having a predisposition to develop such a disease or disorder, then the particular disease or disorder or predisposition for a disease or disorder can be diagnosed, have its prognosis determined, be screened for, or be monitored by detecting decreased levels of the one or more protein complexes, the mRNA that encodes one or more members of the particular one or more protein complexes, or by detecting decreased protein complex functional activity.

Accordingly, in a specific embodiment of the invention, diseases and disorders involving decreased levels of one or more protein complexes can be diagnosed, or their suspected presence can be screened for, or a predisposition to develop such disorders can be detected, by detecting decreased levels of the one or more protein complexes, the mRNA encoding one or more members of the one or more complexes, or complex functional activity, or by detecting mutations in the component proteins that decrease complex formation, e.g., mutations such as translocations in nucleic acids, truncations in the gene or protein, changes in nucleotide or amino acid sequence relative to wild-type, that decrease complex formation.

Accordingly, in a specific embodiment of the invention, diseases and disorders involving aberrant compositions of the complexes can be diagnosed, or their suspected presence can be screened for, or a predisposition to develop such disorders can be detected, by detecting the component proteins of one or more complexes, or the mRNA encoding the members of the one or more complexes.

The use of detection techniques, especially those involving antibodies against a protein complex, provides a method of detecting specific cells that express the complex or component proteins. Using such assays, specific cell types can be defined in which one or more particular protein complexes are expressed, and the presence of the complex or component proteins can be correlated with cell viability, state, health, etc.

Also embodied are methods to detect a protein complex of the present invention in cell culture models that express particular protein complexes or derivatives thereof, for the purpose of characterizing or preparing the complexes for harvest. This embodiment includes cell sorting of prokaryotes such as but not restricted to bacteria (Davey and Kell, 1996, Microbiol. Rev. 60:641-696), primary cultures and tissue specimens from eukaryotes, including mammalian species such as human (Steele et al., 1996, Clin. Obstet. Gynecol 39:801-813), and continuous cell cultures (Orfao and Ruiz-Arguelles, 1996, Clin. Biochem. 29:5-9). Such isolations can be used as methods of diagnosis, described, supra.

In a further specific embodiment, a modulation of the formation process of a complex can be determined.

Such a modulation can either be a change in the typical time course of its formation or a change in the typical steps leading to the formation of the complete complex. Such changes can for example be detected by analysing and comparing the process of complex formation in untreated wild type cells of a particular type and/or cells showing or having the predisposition to develop a certain disease phenotype and/or cells which have been treated with particular conditions and/or particular agents in a particular situation. Methods to study such changes in time course are well known in the art and include for example Westem-blot analysis of the proteins in the complex isolated at different steps of its formation.

Furthermore an aberrant intracellular localization of the protein complex and/or an abberant transcription level of a gene dependent on the complex and/or the abundance and/or activity of a protein or protein complex dependent on the function of the complex and/or a gene dependent on the complex can serve as a marker for a disease and thus have diagnostic utility for any disease which is caused by an aberrant activity, function, composition or formation of the complex of the invention. Methods to study the intracellular localization are well known in the art and include, but are not limited to immunofluorescence analysis using antibodies specific for components of the protein. Preferentially, double-stainings including staining of other cellular structures are being used to facilitate the detection of the intracellular localization. Methods to analyse the transcription levels of a gene dependent on the complex are also well known in the art and include Northern blot analysis, quantitative PCR etc. The abundance of proteins dependent on the protein can be analyzed as described supra. Methods to study changes in the activity of proteins dependent on complex depend on the protein. The choice of such methods will be apparent to any person skilled in the art.

### 4.4. Therapeutic Uses of Protein Complexes/Proteins of the Invention

The present invention is directed to a method for treatment or prevention of various diseases and disorders by administration of a therapeutic compound (termed herein “Therapeutic”). Such “Therapeutics” include, but are not limited to, a protein complex of the present invention, the individual component proteins, and analogs and derivatives (including fragments) of the foregoing (e.g., as described hereinabove); antibodies thereto (as described hereinabove); nucleic acids encoding the component protein, and analogs or derivatives, thereof (e.g., as described hereinabove); component protein antisense nucleic acids, and agents that modulate complex formation and/or activity (i.e., agonists and antagonists).
The physiological role of the complexes as identified herein has remained elusive so far.

However, the protein complexes as identified herein are implicated in processes which are implicated in or associated with pathological conditions.
Diseases and disorders which can be treated and/or prevented and/or diagnosed by Therapeutics interacting with any of the complexes provided herein are for example neurodegenerative diseases such as Alzheimers disease and disorders caused by defects in the Notch-pathway.
These disorders are treated or prevented by administration of a Therapeutic that modulates (i.e. inhibits or promotes) protein complex activity or formation or modulates its function or composition. Diseases or disorders associated with aberrant levels of complex activity or formation, or aberrant levels or activity of the component proteins, or aberrant complex composition or a change in the function, may be treated by administration of a Therapeutic that modulates complex formation or activity or by the administration of a protein complex.
Therapeutic may also be administered to modulate complex formation or activity or level thereof in a microbial organism such as yeast, fungi such as candida albicans causing an infectious disease in animals or humans.

Diseases and disorders characterized by increased (relative to a subject not suffering from the disease or disorder) complex levels or activity can be treated with Therapeutics that antagonize (i.e., reduce or inhibit) complex formation or activity. Therapeutics that can be used include, but are not limited to, the component proteins or an analog, derivative or fragment of the component protein; anti-complex antibodies (e.g., antibodies specific for the protein complex, or a fragment or derivative of the antibody containing the binding region thereof; nucleic acids encoding the component proteins; antisense nucleic acids complementary to nucleic acids encoding the component proteins; and nucleic acids encoding the component protein that are dysfunctional due to, e.g., a heterologous insertion within the protein coding sequence, that are used to “knockout” endogenous protein function by homologous recombination, see, e.g., Capecchi, 1989, Science 244:1288-1292. In one embodiment, a Therapeutic is 1, 2 or more antisense nucleic acids which are complementary to 1, 2, or more nucleic acids, respectfully, that encode component proteins of a complex.

In a specific embodiment of the present invention, a nucleic acid containing a portion of a component protein gene in which gene sequences flank (are both 5′ and 3′ to) a different gene sequence, is used as a component protein antagonist, or to promote component protein inactivation by homologous recombination (see also, Koller and Smithies, 1989, Proc. Natl. Acad. Sci. USA 86:8932-8935; Zijlstra et al., 1989, Nature 342: 435-438). Additionally, mutants or derivatives of a component protein that has greater affinity for another component protein or the complex than wild type may be administered to compete with wild type protein for binding, thereby reducing the levels of complexes containing the wild type protein. Other Therapeutics that inhibit complex function can be identified by use of known convenient in vitro assays, e.g., based on their ability to inhibit complex formation, or as described in further below.

In specific embodiments, Therapeutics that antagonize complex formation or activity are administered therapeutically, including prophylactically, (1) in diseases or disorders involving an increased (relative to normal or desired) level of a complex, for example, in patients where complexes are overactive or overexpressed; or (2) in diseases or disorders where an in vitro (or in vivo) assay (see infra) indicates the utility of antagonist administration. Increased levels of a complex can be readily detected, e.g., by quantifying protein and/or RNA, by obtaining a patient tissue sample (e.g., from biopsy tissue) and assaying it in vitro for RNA or protein levels, or structure and/or activity of the expressed complex (or the encoding mRNA). Many methods standard in the art can be thus employed including, but not limited to, immunoassays to detect complexes and/or visualize complexes (e.g., Western blot analysis, immunoprecipitation followed by sodium dodecyl sulfate polyacrylamide gel electrophoresis [SDS-PAGE], immunocytochemistry, etc.), and/or hybridization assays to detect concurrent expression of component protein mRNA (e.g., Northern assays, dot blot analysis, in situ hybridization, etc.).

A more specific embodiment of the present invention is directed to a method of reducing complex expression (i.e., expression of the protein components of the complex and/or formation of the complex) by targeting mRNAs that express the protein moieties. RNA therapeutics currently fall within three classes, antisense species, ribozymes, or RNA aptamers (Good et al., 1997, Gene Therapy 4:45-54).

Antisense oligonucleotides have been the most widely used. By way of example, but not limitation, antisense oligonucleotide methodology to reduce complex formation is presented below.

Ribozyme therapy involves the administration, induced expression, etc. of small RNA molecules with enzymatic ability to cleave, bind, or otherwise inactivate specific RNAs, to reduce or eliminate expression of particular proteins (Grassi and Marini, 1996, Annals of Medicine 28:499-510; Gibson, 1996, Cancer and Metastasis Reviews 15:287-299). RNA aptamers are specific RNA ligand proteins, such as for Tat and Rev RNA (Good et al., 1997, Gene Therapy 4:45-54) that can specifically inhibit their translation. Aptamers specific for component proteins can be identified by many methods well known in the art, for example, by affecting the formation of a complex in the protein-protein interaction assay described below.

In another embodiment, the activity or levels of a component protein are reduced by administration of another component protein, or the encoding nucleic acid, or an antibody that immunospecifically binds to the component protein, or a fragment or a derivative of the antibody containing the binding domain thereof.

In another aspect of the invention, diseases or disorders associated with increased levels of an component protein of the complex may be treated or prevented by administration of a Therapeutic that increases complex formation if the complex formation acts to reduce or inactivate the component protein through complex formation. Such diseases or disorders can be treated or prevented by administration of one component member of the complex, administration of antibodies or other molecules that stabilize the complex, etc.

Diseases and disorders associated with underexpression of a complex, or a component protein, are treated or prevented by administration of a Therapeutic that promotes (i.e., increases or supplies) complex levels and/or function, or individual component protein function. Examples of such a Therapeutic include but are not limited to a complex or a derivative, analog or fragment of the complex that are functionally active (e.g., able to form a complex), un-complexed component proteins and derivatives, analogs, and fragments of un-complexed component proteins, and nucleic acids encoding the members of a complex or functionally active derivatives or fragments of the members of the complex, e.g., for use in gene therapy. In a specific embodiment, a Therapeutic includes derivatives, homologs or fragments of a component protein that increase and/or stabilize complex formation. Examples of other agonists can be identified using in vitro assays or animal models, examples of which are described further below.

In yet other specific embodiments of the present invention, Therapeutics that promote complex function are administered therapeutically, including prophylactically, (1) in diseases or disorders involving an absence or decreased (relative to normal or desired) level of a complex, for example, in patients where a complex, or the individual components necessary to form the complex, is lacking, genetically defective, biologically inactive or underactive, or under-expressed; or (2) in diseases or disorders wherein an in vitro or in vivo assay indicates the utility of complex agonist administration. The absence or decreased level of a complex, component protein or function can be readily detected, e.g., by obtaining a patient tissue sample (e.g., from biopsy tissue) and assaying it in vitro for RNA or protein levels, structure and/or activity of the expressed complex and/or the concurrent expression of mRNA encoding the two components of the complex. Many methods standard in the art can be thus employed, including but not limited to immunoassays to detect and/or visualize a complex, or the individual components of a complex (e.g., Western blot analysis, immunoprecipitation followed by sodium dodecyl sulfate polyacrylamide gel electrophoresis [SDS-PAGE], immunocytochemistry, etc.) and/or hybridization assays to detect expression of mRNAs encoding the individual protein components of a complex by detecting and/or visualizing component mRNA concurrently or separately using, e.g., Northern assays, dot blot analysis, in situ hybridization, etc.

In specific embodiments, the activity or levels of a component protein are increased by administration of another component protein of the same complex, or a derivative, homolog or analog thereof, a nucleic acid encoding the other component, or an agent that stabilizes or enhances the other component, or a fragment or derivative of such an agent.

Generally, administration of products of species origin or species reactivity (in the case of antibodies) that is the same species as that of the patient is preferred. Thus, in a preferred embodiment, a human complex, or derivative, homolog or analog thereof; nucleic acids encoding the members of the human complex or a derivative, homolog or analog thereof; an antibody to a human complex, or a derivative thereof; or other human agents that affect component proteins or the complex, are therapeutically or prophylactically administered to a human patient.

Preferably, suitable in vitro or in vivo assays are utilized to determine the effect of a specific Therapeutic and whether its administration is indicated for treatment of the affected tissue or individual.

In various specific embodiments, in vitro assays can be carried out with representative cells of cell types involved in a patient's disorder, to determine if a Therapeutic has a desired effect upon such cell types.

Compounds for use in therapy can be tested in suitable animal model systems prior to testing in humans, including, but not limited to, rats, mice, chicken, cows, monkeys, rabbits, etc. For in vivo testing, prior to administration to humans, any animal model system known in the art may be used. Additional descriptions and sources of Therapeutics that can be used according to the invention are found in Sections 4.1 to 4.3 and 4.7 herein.

### 4.4.1. Gene Therapy

In a specific embodiment of the present invention, nucleic acids comprising a sequence encoding the component proteins, or a functional derivative thereof, are administered to modulate complex activity or formation by way of gene therapy. Gene therapy refers to therapy performed by the administration of a nucleic acid to a subject. In this embodiment of the present invention, the nucleic acid expresses its encoded protein(s) that mediates a therapeutic effect by modulating complex activity or formation. Any of the methods for gene therapy available in the art can be used according to the present invention. Exemplary methods are described below.

For general reviews of the methods of gene therapy, see Goldspiel et al.,1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; Morgan and Anderson, 1993, Ann. Rev. Biochem. 62:191-217; and May, 1993, TIBTECH 11:155-215. Methods commonly known in the art of recombinant DNA technology which can be used are described in Ausubel et al., eds., 1993, Current Protocols in Molecular Biology, John Wiley & Sons, NY; and Kriegler, 1990, Gene Transfer and Expression, A Laboratory Manual, Stockton Press, NY.

In a preferred aspect, the Therapeutic comprises a nucleic acid that is part of an expression vector that expresses one or more of the component proteins, or fragments or chimeric proteins thereof, in a suitable host. In particular, such a nucleic acid has a promoter operably linked to the protein coding region(s) (or, less preferably separate promoters linked to the separate coding regions separately), said promoter being inducible or constitutive, and optionally, tissue-specific. In another particular embodiment, a nucleic acid molecule is used in which the coding sequences, and any other desired sequences, are flanked by regions that promote homologous recombination at a desired site in the genome, thus providing for intra-chromosomal expression of the component protein nucleic acids (Koller and Smithies, 1989, Proc. Natl. Acad. Sci. USA 86:8932-8935; Zijlstra et al., 1989, Nature 342:435-438).

Delivery of the nucleic acid into a patient may be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (U.S. Pat. No. 4,980,286), or by direct injection of naked DNA, or by use of microparticle bombardment (e.g., a gene gun; Biolistic, Dupont), or coating with lipids or cell-surface receptors, or through use of transfecting agents, by encapsulation in liposomes, microparticles, or microcapsules, or by administering it in linkage to a peptide that is known to enter the nucleus, or by administering it in linkage to a ligand subject to receptor-mediated endocytosis that can be used to target cell types specifically expressing the receptors (e.g., Wu and Wu, 1987, J. Biol. Chem. 262:4429-4432), etc. In another embodiment, a nucleic acid-ligand complex can be formed in which the ligand comprises a fusogenic viral peptide that disrupts endosomes, allowing the nucleic acid to avoid lysosomal degradation. In yet another embodiment, the nucleic acid can be targeted in vivo for cell specific uptake and expression, by targeting a specific receptor (see, e.g., International Patent Publications WO 92/06180; WO 92/22635; WO 92/20316; WO 93/14188; and WO 93/20221. Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a specific embodiment, a viral vector that contains the component protein encoding nucleic acids is used. For example, a retroviral vector can be used (Miller et al., 1993, Meth. Enzymol. 217:581-599). These retroviral vectors have been modified to delete retroviral sequences that are not necessary for packaging of the viral genome and integration into host cell DNA. The encoding nucleic acids to be used in gene therapy is/are cloned into the vector, which facilitates delivery of the gene into a patient. More detail about retroviral vectors can be found in Boesen et al., 1994, Biotherapy 6:291-302, which describes the use of a retroviral vector to deliver the mdr1 gene to hematopoetic stem cells in order to make the stem cells more resistant to chemotherapy. Other references illustrating the use of retroviral vectors in gene therapy are Clowes et al., 1994, J. Clin. Invest. 93:644-451; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are the liver, the central nervous system, endothelial cells and muscle. Adenoviruses have the advantage of being capable of infecting non-dividing cells. Kozarsky and Wilson, 1993, Current Opinion in Genetics and Development 3:499-503, discuss adenovirus-based gene therapy. The use of adenovirus vectors to transfer genes to the respiratory epithelia of rhesus monkeys has been demonstrated by Bout et al., 1994, Human Gene Therapy 5:3-10. Other instances of the use of adenoviruses in gene therapy can be found in Rosenfeld etal., 1991, Science 252:431-434; Rosenfeld et al., 1992, Cell 68:143-155; and Mastrangeli et al., 1993, J. Clin. Invest. 91:225-234.

Adeno-associated virus (AAV) has also been proposed for use in gene therapy (Walsh et al., 1993, Proc. Soc. Exp. Biol. Med. 204:289-300.

Another approach to gene therapy involves transferring a gene into cells in tissue culture by methods such as electroporation, lipofection, calcium phosphate-mediated transfection, or viral infection. Usually, the method of transfer includes the transfer of a selectable marker to the cells. The cells are then placed under selection to isolate those cells that have taken up and are expressing the transferred gene from these that have not. Those cells are then delivered to a patient.

In this embodiment, the nucleic acid is introduced into a cell prior to administration in vivo of the resulting recombinant cell. Such introduction can be carried out by any method known in the art including, but not limited to, transfection by electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably, is heritable and expressible by its cell progeny.

The resulting recombinant cells can be delivered to a patient by various methods known in the art. In a preferred embodiment, epithelial cells are injected, e.g., subcutaneously. In another embodiment, recombinant skin cells may be applied as a skin graft onto the patient. Recombinant blood cells (e.g., hematopoetic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes, blood cells such as T lymphocytes, B lymphocytes, monocytes, macrophages, neutrophils, eosinophils, megakaryocytes, and granulocytes, various stem or progenitor cells, in particular hematopoetic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, a component protein encoding nucleic acid is/are introduced into the cells such that the gene or genes are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention. Such stem cells include but are not limited to hematopoetic stem cells (HSCs), stem cells of epithelial tissues such as the skin and the lining of the gut, embryonic heart muscle cells, liver stem cells (International Patent Publication WO 94/08598), and neural stem cells (Stemple and Anderson, 1992, Cell 71:973-985).

Epithelial stem cells (ESCs), or keratinocytes, can be obtained from tissues such as the skin and the lining of the gut by known procedures (Rheinwald, 1980, Meth. Cell Biol. 2A:229). In stratified epithelial tissue such as the skin, renewal occurs by mitosis of stem cells within the germinal layer, the layer closest to the basal lamina. Similarly, stem cells within the lining of the gut provide for a rapid renewal rate of this tissue. ESCs or keratinocytes obtained from the skin or lining of the gut of a patient or donor can be grown in tissue culture (Rheinwald, 1980, Meth. Cell Bio. 2A:229; Pittelkow and Scott, 1986, Mayo Clinic Proc. 61:771). If the ESCs are provided by a donor, a method for suppression of host versus graft reactivity (e.g., irradiation, or drug or antibody administration to promote moderate immunosuppression) can also be used.

With respect to hematopoetic stem cells (HSCs), any technique that provides for the isolation, propagation, and maintenance in vitro of HSCs can be used in this embodiment of the invention. Techniques by which this may be accomplished include (a) the isolation and establishment of HSC cultures from bone marrow cells isolated from the future host, or a donor, or (b) the use of previously established long-term HSC cultures, which may be allogeneic or xenogeneic. Non-autologous HSCs are used preferably in conjunction with a method of suppressing transplantation immune reactions between the future host and patient. In a particular embodiment of the present invention, human bone marrow cells can be obtained from the posterior iliac crest by needle aspiration (see, e.g., Kodo et al., 1984, J. Clin. Invest. 73: 1377-1384). In a preferred embodiment of the present invention, the HSCs can be made highly enriched or in substantially pure form. This enrichment can be accomplished before, during, or after long-term culturing, and can be done by any technique known in the art. Long-term cultures of bone marrow cells can be established and maintained by using, for example, modified Dexter cell culture techniques (Dexter et al., 1977, J. Cell Physiol. 91:335) or Witlock-Witte culture techniques (Witlock and Witte, 1982, Proc. Natl. Acad. Sci. USA 79:3608-3612).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

Additional methods can be adapted for use to deliver a nucleic acid encoding the component proteins, or functional derivatives thereof, e.g., as described in Section 4.1,

### 4.4.2. Use of Antisense Oligonucleotides for Suppresson of Protein Comples Formation or Protein Complex/Protein Activity

In a specific embodiment of the present invention, protein complex activity and formation and protein activity is inhibited by use of antisense nucleic acids for the component proteins of the complex, that inhibit transcription and/or translation of their complementary sequence. The present invention provides the therapeutic or prophylactic use of nucleic acids of at least six nucleotides that are antisense to a gene or cDNA encoding a component protein, or a portion thereof. An “antisense” nucleic acid as used herein refers to a nucleic acid capable of hybridizing to a sequence-specific portion of a component protein RNA (preferably mRNA) by virtue of some sequence complementarity. The antisense nucleic acid may be complementary to a coding and/or noncoding region of a component protein mRNA. Such antisense nucleic acids that inhibit complex formation or activity have utility as Therapeutics, and can be used in the treatment or prevention of disorders as described supra.

The antisense nucleic acids of the invention can be oligonucleotides that are double-stranded or single-stranded, RNA or DNA, or a modification or derivative thereof, which can be directly administered to a cell, or which can be produced intracellularly by transcription of exogenous, introduced sequences.

In another embodiment, the present invention is directed to a method for inhibiting the expression of component protein nucleic acid sequences, in a prokaryotic or eukaryotic cell, comprising providing the cell with an effective amount of a composition comprising an antisense nucleic acid of the component protein, or a derivative thereof, of the invention.

The antisense nucleic acids are of at least six nucleotides and are preferably oligonucleotides, ranging from 6 to about 200 nucleotides. In specific aspects, the oligonucleotide is at least 10 nucleotides, at least 15 nucleotides, at least 100 nucleotides, or at least 200 nucleotides. The oligonucleotides can be DNA or RNA or chimeric mixtures, or derivatives or modified versions thereof, and either single-stranded or double-stranded. The oligonucleotide can be modified at the base moiety, sugar moiety, or phosphate backbone. The oligonucleotide may include other appending groups such as peptides, agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; International Patent Publication No. WO 88/09810) or blood-brain barrier (see, e.g., International Patent Publication No. WO 89/10134), hybridization-triggered cleavage agents (see, e.g., Krol et al., 1988, BioTechniques 6:958-976), or intercalating agents (see, e.g., Zon, 1988, Pharm. Res. 5:539-549).

In a preferred aspect of the invention, an antisense oligonucleotide is provided, preferably as single-stranded DNA. The oligonucleotide may be modified at any position in its structure with constituents generally known in the art.

The antisense oligonucleotides may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thio-uridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5N-methoxycarboxymethyluracil, 5-methoxyuracil, 2-methyl-thio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

In another embodiment, the oligonucleotide comprises at least one modified sugar moiety selected from the group including, but not limited to, arabinose, 2-fluoroarabinose, xylulose, and hexose.

In yet another embodiment, the oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal, or an analog of the foregoing.

In yet another embodiment, the oligonucleotide is a 2-a-anomeric oligonucleotide. An a-anomeric oligonucleotide forms specific double-stranded hybrids with complementary RNA in which, contrary to the usual β-units, the strands run parallel to each other (Gautier et al., 1987, Nucl. Acids Res. 15:6625-6641).

The oligonucleotide may be conjugated to another molecule, e.g., a peptide, hybridization-triggered cross-linking agent, transport agent, hybridization-triggered cleavage agent, etc.

Oligonucleotides of the invention may be synthesized by standard methods known in the art, e.g., by use of an automated DNA synthesizer (such as are commercially avail-able from Biosearch, Applied Biosystems, etc.). As examples, phosphorothioate oligo-nucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

In a specific embodiment, the antisense oligonucleotides comprise catalytic RNAs, or ribozymes (see, e.g., International Patent Publication No. WO 90/11364; Sarver et al., 1990, Science 247:1222-1225). In another embodiment, the oligonucleotide is a 2′-0-methylribonucleotide (Inoue et al., 1987, Nucl. Acids Res. 15:6131-6148), or a chimeric RNA-DNA analog (Inoue et al., 1987, FEBS Lett. 215:327-330).

In an alternative embodiment, the antisense nucleic acids of the invention are produced intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding the component protein. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art to be capable of replication and expression in mammalian cells. Expression of the sequences encoding the antisense RNAs can be by any promoter known in the art to act in mammalian, preferably human, cells. Such promoters can be inducible or constitutive. Such promoters include, but are not limited to, the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), etc.

The antisense nucleic acids of the invention comprise a sequence complementary to at least a portion of an RNA transcript of a component protein gene, preferably a human gene. However, absolute complementarity, although preferred, is not required. A sequence “complementary to at least a portion of an RNA,” as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a component protein RNA it may contain and still form a stable duplex (or triplex, as the case may be). One skilled in the art can ascertain a tolerable degree of mismatch by use of standard procedures to determine the melting point of the hybridized complex.

The component protein antisense nucleic acids can be used to treat (or prevent) disorders of a cell type that expresses, or preferably overexpresses, a protein complex.

Cell types that express or overexpress component protein RNA can be identified by various methods known in the art. Such methods include, but are not limited to, hybridization with component protein-specific nucleic acids (e.g., by Northern blot hybridization, dot blot hybridization, or in situ hybridization), or by observing the ability of RNA from the cell type to be translated in vitro into the component protein by immunohistochemistry, Western blot analysis, ELISA, etc. In a preferred aspect, primary tissue from a patient can be assayed for protein expression prior to treatment, e.g., by immunocytochemistry, in situ hybridization, or any number of methods to detect protein or mRNA expression.

Pharmaceutical compositions of the invention (see 4.7, ), comprising an effective amount of a protein component antisense nucleic acid in a pharmaceutically acceptable carrier can be administered to a patient having a disease or disorder that is of a type that expresses or overexpresses a protein complex of the present invention.

The amount of antisense nucleic acid that will be effective in the treatment of a particular disorder or condition will depend on the nature of the disorder or condition, and can be determined by standard clinical techniques. Where possible, it is desirable to determine the antisense cytotoxicity in vitro, and then in useful animal model systems, prior to testing and use in humans.

In a specific embodiment, pharmaceutical compositions comprising antisense nucleic acids are administered via liposomes, microparticles, or microcapsules. In various embodiments of the invention, it may be useful to use such compositions to achieve sustained release of the antisense nucleic acids. In a specific embodiment, it may be desirable to utilize liposomes targeted via antibodies to specific identifiable central nervous system cell types (Leonetti et al., 1990, Proc. Natl. Acad. Sci. U.S.A. 87:2448-2451; Renneisen et al., 1990, J. Biol. Chem. 265:16337-16342).

### 4.5. Assays of Protein Complexes/Proteins of the Invention and Derivates and Analogs thereof

The functional activity of a protein complex of the present invention, or a derivative, fragment or analog thereof or protein component thereof, can be assayed by various methods. Potential modulators (e.g., agonists and antagonists) of complex activity or formation, e.g., anti-complex antibodies and antisense nucleic acids, can be assayed for the ability to modulate complex activity or formation.

In one embodiment of the present invention, where one is assaying for the ability to bind or compete with a wild-type complex for binding to an anti-complex antibody, various immunoassays known in the art can be used, including but not limited to competitive and non-competitive assay systems using techniques such as radioimmunoassay, ELISA (enzyme linked immunosorbent assay), “sandwich” immunoassays, immunoradiometric assays, gel diffusion precipitin reactions, immunodiffusion assays, in situ immunoassays (using colloidal gold, enzyme or radioisotope labels), western blot analysis, precipitation reactions, agglutination assays (e.g., gel agglutination assays, hemagglutination assays), complement fixation assays, immunofluorescence assays, protein A assays, immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

The expression of the component protein genes (both endogenous and those expressed from cloned DNA containing the genes) can be detected using techniques known in the art, including but not limited to Southern hybridization (Southern, 1975, J. Mol. Biol. 98:503-517), northern hybridization (see, e.g., Freeman et al., 1983, Proc. Natl. Acad. Sci. USA 80:4094-4098), restriction endonuclease mapping (Sambrook et al., 1989, Molecular Cloning, A Laboratory Manual, 2^{nd} Ed. Cold Spring Harbor Laboratory Press, New York), RNase protection assays (Current Protocols in Molecular Biology, John Wiley and Sons, New York, 1997), DNA sequence analysis, and polymerase chain reaction amplification (PCR; U.S. Pat. Nos. 4,683,202, 4,683,195, and 4,889,818; Gyllenstein et al., 1988, Proc. Natl. Acad. Sci. USA 85:7652-7657; Ochman et al., 1988, Genetics 120:621-623; Loh et al., 1989, Science 243:217-220) followed by Southern hybridization with probes specific for the component protein genes, in various cell types. Methods of amplification other than PCR commonly known in the art can be employed. In one embodiment, Southern hybridization can be used to detect genetic linkage of component protein gene mutations to physiological or pathological states. Various cell types, at various stages of development, can be characterized for their expression of component proteins at the same time and in the same cells. The stringency of the hybridization conditions for northern or Southern blot analysis can be manipulated to ensure detection of nucleic acids with the desired degree of relatedness to the specific probes used. Modifications to these methods and other methods commonly known in the art can be used.

Derivatives (e.g., fragments), homologs and analogs of one component protein can be assayed for binding to another component protein in the same complex by any method known in the art, for example the modified yeast matrix mating test described in Section 4.6.1, immunoprecipitation with an antibody that binds to the component protein complexed with other component proteins in the same complex, followed by size fractionation of the immunoprecipitated proteins (e.g., by denaturing or nondenaturing polyacrylamide gel electrophoresis), Western blot analysis, etc.

One embodiment of the invention provides a method for screening a derivative, homolog or analog of a component protein for biological activity comprising contacting said derivative, homolog or analog of the component protein with the other component proteins in the same complex; and detecting the formation of a complex between said derivative, homolog or analog of the component protein and the other component proteins; wherein detecting formation of said complex indicates that said derivative, homolog or analog of has biological (e.g., binding) activity.

The invention also provides methods of modulating the activity of a component protein that can participate in a protein complex by administration of a binding partner of that protein or derivative, homolog or analog thereof.

In a specific embodiment of the present invention, a protein complex of the present invention is administered to treat or prevent a disease or disorder, since the complex and/or component proteins have been implicated in the disease and disorder. Accordingly, a protein complex or a derivative, homolog, analog or fragment thereof, nucleic acids encoding the component proteins, anti-complex antibodies, and other modulators of protein complex activity, can be tested for activity in treating or preventing a disease or disorder in in vitro and in vivo assays.

In one embodiment, a Therapeutic of the invention can be assayed for activity in treating or preventing a disease by contacting cultured cells that exhibit an indicator of the disease in vitro, with the Therapeutic, and comparing the level of said indicator in the cells contacted with the Therapeutic, with said level of said indicator in cells not so contacted, wherein a lower level in said contacted cells indicates that the Therapeutic has activity in treating or preventing the disease.

In another embodiment of the invention, a Therapeutic of the invention can be assayed for activity in treating or preventing a disease by administering the Therapeutic to a test animal that is predisposed to develop symptoms of a disease, and measuring the change in said symptoms of the disease after administration of said Therapeutic, wherein a reduction in the severity of the symptoms of the disease or prevention of the symptoms of the disease indicates that the Therapeutic has activity in treating or preventing the disease. Such a test animal can be any one of a number of animal models known in the art for disease. These animal models are well known in the art. These animal models include, but are not limited to those which are listed in the section 4.6 as exemplary animald models to study any of the complexes provided in the invention.

### 4.6 Screening for Modulators of the Protein Complexes/Proteins of the Invention

A complex of the present invention, the component proteins of the complex and nucleic acids encoding the component proteins, as well as derivatives and fragments of the amino and nucleic acids, can be used to screen for compounds that bind to, or modulate the amount of, activity of, or protein component composition of, said complex, and thus, have potential use as modulators, i.e., agonists or antagonists, of complex activity, and/or complex formation, i.e., the amount of complex formed, and/or protein component composition of the complex.

Thus, the present invention is also directed to methods for screening for molecules that bind to, or modulate the function of, amount of, activity of, formation of or protein component composition of, a complex of the present invention. In one embodiment of the invention, the method for screening for a molecule that modulates directly or indirectly the function, activity or formation of a complex of the present invention comprises exposing said complex, or a cell or organism containing the complex machinery, to one or more candidate molecules under conditions conducive to modulation; and determining the amount of, the biochemical activity of (preferentially gamma-secretase activity), protein components of, and/or intracellular localization of, said complex and/or the transcription level of a gene dependend on the complex and/or the abundance and/or activity of a protein or protein complex dependend on the function of the complex and/or product of a gene dependent on the complex in the presence of the one or more candidate molecules, wherein a change in said amount, activity, protein components or intracellular localization relative to said amount, activity, protein components and/or intracellular localization and/or a change in the transcription level of a gene dependend on the complex and/or the abundance and/or activity of a protein or protein complex dependent on the function of the complex and/or product of a gene dependent on the complex in the absence of said candidate molecules indicates that the molecule modulates function, activity or composition of said complex.

In a further specific embodiment, a modulation of the formation process of a complex can be determined.

Such a modulation can either be a change in the typical time course of its formation or a change in the typical steps leading to the formation of the complete complex. Such changes can for example be detected by analysing and comparing the process of complex formation in untreated wild type cells of a particular type and/or cells showing or having the predisposition to develop a certain disease phenotype and/or cells which have been treated with particular conditions and/or particular agents in a particular situation. Methods to study such changes in time course are well known in the art and include for example Western-blot analysis of the proteins in the complex isolated at different steps of its formation.

Furthermore an aberrant intracellular localization of the protein complex and/or an abberant transcription level of a gene dependent on the complex and/or the abundance and/or activity of a protein or protein complex dependent on the function of the complex and/or a gene dependent on the complex can serve as a marker for a disease and thus have diagnostic utility for any disease which is caused by an aberrant activity, function, composition or formation of the complex of the invention. Methods to study the intracellular localization are well known in the art and include, but are not limited to immunofluorescence analysis using antibodies specific for components of the protein. Preferentially, double-stainings including staining of other cellular structures are being used to facilitate the detection of the intracellular localization. Methods to analyse the transcription levels of a gene dependent on the complex are also well known in the art and include Northern blot analysis, quantitative PCR etc. The abundance of proteins dependent on the protein can be analyzed as described supra. Methods to study changes in the activity of proteins dependent on complex depend on the protein. The choice of such methods will be apparent to any person skilled in the art.

In another embodiment, the present invention further relates to a process for the identification and/or preparation of an effector of the the complexes provided herein cleavage comprising the step of bringing into contact a product of any of claims 1 to 8 with a compound, a mixture or a library of compounds and determining whether the compound or a certain compound of the mixture or library binds to the product and/or effects the products biological activity and optionally further purifying the compound positively tested as effector.

In another embodiment, the present invention is directed to a method for screening for a molecule that binds a protein complex of the present invention comprising exposing said complex, or a cell or organism containing the complex machinery, to one or more candidate molecules; and determining whether said complex is bound by any of said candidate molecules. Such screening assays can be carried out using cell-free and cell-based methods that are commonly known in the art in vitro, in vivo or ex vivo. For example, an isolated complex can be employed, or a cell can be contacted with the candidate molecule and the complex can be isolated from such contacted cells and the isolated complex can be assayed for activity or component composition. In another example, a cell containing the complex can be contacted with the candidate molecule and the levels of the complex in the contacted cell can be measured.

For example, assays can be carried out using recombinant cells expressing the protein components of a complex, to screen for molecules that bind to, or interfere with, or promote complex activity or formation. In preferred embodiments, polypeptide derivatives that have superior stabilities but retain the ability to form a complex (e.g., one or more component proteins modified to be resistant to proteolytic degradation in the binding assay buffers, or to be resistant to oxidative degradation), are used to screen for modulators of complex activity or formation. Such resistant molecules can be generated, e.g., by substitution of amino acids at proteolytic cleavage sites, the use of chemically derivatized amino acids at proteolytic susceptible sites, and the replacement of amino acid residues subject to oxidation, i.e. methionine and cysteine.

A particular aspect of the present invention relates to identifying molecules that inhibit or promote formation or degradation of a complex of the present invention, In another embodiment of the invention, a modulator is identified by administering a candidate molecule to a transgenic non-human animal expressing the complex component proteins from promoters that are not the native promoters of the respective proteins, more preferably where the candidate molecule is also recombinantly expressed in the transgenic non-human animal. Alternatively, the method for identifying such a modulator can be carried out in vitro, preferably with a purified complex, and a purified candidate molecule.

Agents/molecules (candidate molecules) to be screened can be provided as mixtures of a limited number of specified compounds, or as compound libraries, peptide libraries and the like. Agents/molecules to be screened may also include all forms of antisera, antisense nucleic acids, etc., that can modulate complex activity or formation. Exemplary candidate molecules and libraries for screening are set forth in Section 4.6.1,

Screening the libraries can be accomplished by any of a variety of commonly known methods. See, e.g., the following references, which disclose screening of peptide libraries: Parmley and Smith, 1989, Adv. Exp. Med. Biol. 251:215-218; Scott and Smith, 1990, Science 249:386-390; Fowlkes et al., 1992, BioTechniques 13:422-427; Oldenburg et al., 1992, Proc. Natl. Acad. Sci. USA 89:5393-5397; Yu et al., 1994, Cell 76:933-945; Staudt et al., 1988, Science 241:577-580; Bock et al., 1992, Nature 355:564-566; Tuerk et al., 1992, Proc. Natl. Acad. Sci. USA 89:6988-6992; Ellington et al., 1992, Nature 355:850-852; U.S. Pat. No. 5,096,815, U.S. Pat. No. 5,223,409, and U.S. Pat. No. 5,198,346, all to Ladner et al.; Rebar and Pabo, 1993, Science 263:671-673; and International Patent Publication No. WO 94/18318.

In a specific embodiment, screening can be carried out by contacting the library members with a complex immobilized on a solid phase, and harvesting those library members that bind to the protein (or encoding nucleic acid or derivative). Examples of such screening methods, termed “panning” techniques, are described by way of example in Parmley and Smith, 1988, Gene 73:305-318; Fowlkes et al., 1992, BioTechniques 13:422-427; International Patent Publication No. WO 94/18318; and in references cited hereinabove.

In a specific embodiment, fragments and/or analogs of protein components of a complex, especially peptidomimetics, are screened for activity as competitive or non-competitive inhibitors of complex formation (amount of complex or composition of complex) or activity in the cell, which thereby inhibit complex activity or formation in the cell.

In one embodiment, agents that modulate (i.e., antagonize or agonize) complex activity or formation can be screened for using a binding inhibition assay, wherein agents are screened for their ability to modulate formation of a complex under aqueous, or physiological, binding conditions in which complex formation occurs in the absence of the agent to be tested. Agents that interfere with the formation of complexes of the invention are identified as antagonists of complex formation. Agents that promote the formation of complexes are identified as agonists of complex formation. Agents that completely block the formation of complexes are identified as inhibitors of complex formation.

Methods for screening may involve labeling the component proteins of the complex with radioligands (e.g., ¹²⁵I or ³H), magnetic ligands (e.g., paramagnetic beads covalently attached to photobiotin acetate), fluorescent ligands (e.g., fluorescein or rhodamine), or enzyme ligands (e.g., luciferase or beta-galactosidase). The reactants that bind in solution can then be isolated by one of many techniques known in the art, including but not restricted to, co-immunoprecipitation of the labeled complex moiety using antisera against the unlabeled binding partner (or labeled binding partner with a distinguishable marker from that used on the second labeled complex moiety), immunoaffinity chromatography, size exclusion chromatography, and gradient density centrifugation. In a preferred embodiment, the labeled binding partner is a small fragment or peptidomimetic that is not retained by a commercially available filter. Upon binding, the labeled species is then unable to pass through the filter, providing for a simple assay of complex formation.

Methods commonly known in the art are used to label at least one of the component members of the complex. Suitable labeling methods include, but are not limited to, radiolabeling by incorporation of radiolabeled amino acids, e.g., ³H-leucine or ³⁵S-methionine, radiolabeling by post-translational iodination with ¹²⁵I or ¹³¹I using the chloramine T method, Bolton-Hunter reagents, etc., or labeling with ³²P using phosphorylase and inorganic radiolabeled phosphorous, biotin labeling with photobiotin-acetate and sunlamp exposure, etc. In cases where one of the members of the complex is immobilized, e.g., as described infra, the free species is labeled. Where neither of the interacting species is immobilized, each can be labeled with a distinguishable marker such that isolation of both moieties can be followed to provide for more accurate quantification, and to distinguish the formation of homomeric from heteromeric complexes. Methods that utilize accessory proteins that bind to one of the modified interactants to improve the sensitivity of detection, increase the stability of the complex, etc., are provided.

Typical binding conditions are, for example, but not by way of limitation, in an aqueous salt solution of 10-250 mM NaCl, 5-50 mM Tris-HCl, pH 5-8, and 0.5% Triton X-100 or other detergent that improves specificity of interaction. Metal chelators and/or divalent cations may be added to improve binding and/or reduce proteolysis. Reaction temperatures may include 4, 10, 15, 22, 25, 35, or 42 degrees Celsius, and time of incubation is typically at least 15 seconds, but longer times are preferred to allow binding equilibrium to occur. Particular complexes can be assayed using routine protein binding assays to determine optimal binding conditions for reproducible binding.

The physical parameters of complex formation can be analyzed by quantification of complex formation using assay methods specific for the label used, e.g., liquid scintillation counting for radioactivity detection, enzyme activity for enzyme-labeled moieties, etc. The reaction results are then analyzed utilizing Scatchard analysis, Hill analysis, and other methods commonly known in the arts (see, e.g., Proteins, Structures, and Molecular Principles, 2^{nd} Edition (1993) Creighton, Ed., W. H. Freeman and Company, New York).

In a second common approach to binding assays, one of the binding species is immobilized on a filter, in a microtiter plate well, in a test tube, to a chromatography matrix, etc., either covalently or non-covalently. Proteins can be covalently immobilized using any method well known in the art, for example, but not limited to the method of Kadonaga and Tjian, 1986, Proc. Natl. Acad. Sci. USA 83:5889-5893, i.e., linkage to a cyanogen-bromide derivatized substrate such as CNBr-Sepharose 4B (Pharmacia). Where needed, the use of spacers can reduce steric hindrance by the substrate. Non-covalent attachment of proteins to a substrate include, but are not limited to, attachment of a protein to a charged surface, binding with specific antibodies, binding to a third unrelated interacting protein, etc.

Assays of agents (including cell extracts or a library pool) for competition for binding of one member of a complex (or derivatives thereof) with another member of the complex labeled by any means (e.g., those means described above) are provided to screen for competitors or enhancers of complex formation.

In specific embodiments, blocking agents to inhibit non-specific binding of reagents to other protein components, or absorptive losses of reagents to plastics, immobilization matrices, etc., are included in the assay mixture. Blocking agents include, but are not restricted to bovine serum albumin, beta-casein, nonfat dried milk, Denhardt's reagent, Ficoll, polyvinylpyrolidine, nonionic detergents (NP40, Triton X-100, Tween 20, Tween 80, etc.), ionic detergents (e.g., SDS, LDS, etc.), polyethylene glycol, etc. Appropriate blocking agent concentrations allow complex formation.

After binding is performed, unbound, labeled protein is removed in the supernatant, and the immobilized protein retaining any bound, labeled protein is washed extensively. The amount of bound label is then quantified using standard methods in the art to detect the label as described, supra.

In another specific embodiments screening for modulators of the protein complexes/protein as provided herein can be carried out by attaching those and/or the antibodies as provided herein to a solid carrier. In a further specific embodiment, the invention relates to an array of said molecules.

The preparation of such an array containing different types of proteins, including antibodies) is well known in the art and is apparent to a person skilled in the art (see e.g. Ekins R P et. al. (1989) Journal of Pharmaceutical and Biomedical Analysis 7:155-168; Mitchell P et. al. (2002) Nature Biotechnology 20: 225-229; Petricoin E F et. al. (2002) Lancet 359: 572-577; Templin M F et. al. (2001) Trends in Biotechnology 20:160-166; Wilson D S and Nock S (2001) Curr Opin Chem Biol 6: 81-85; Lee K B et. al. (2002) Science 295: 1702-1705; MacBeath G and Schreiber S. L (2000) Science 289, 1760; Blawas A S and Reichert W M (1998) Biomaterials 19, 595; Kane R S et. al. (1999) Biomaterials 20: 2363; Chen C S et al. (1997) Science 276, 1425; Vaugham T J et. al (1996) Nature Biotechnol 14: 309-314; Mahler S M et al. (1997) Immunotechnology 3, 31-43; Roberts et al. (1999) Curr Opin Chem Biol 3: 268-273; Nord K et al. (1997) Nature Biotechlol 15: 772-777; Nord K et al. (2001) Eur J Biochem 268: 4269-4277; Brody E and Gold L (2000) Rev Mol Biotechlol 74: 5-13; Karlstroem A and Nygren P A (2001) Anal Biochem 295, 22-30; Nelson R W et. al. (2000) Electrophoresis 21: 1155-1163. Honore B et al. (2001) Expert Rev Mol Diagn 3: 265-274, Albala J S (2001) Expert Rev Mol Diagn 2:145-152, Figeys D and Pinto D (2001) Electrophoresis 2: 208-16 and references in the publications listed here.).

Complexes can be attached to an array by different means as will be apparent to a person skilled in the art. Complexes can for example be added to the array via a TAP-tag (as described in WO/0009716 and in Rigaut, G et.al. (1999), Nature Biotechnology. Vol 17 (10): 1030-1032 ) after the purification step or by another suitable purification scheme as will be apparent to a person skilled in the art.

Optionally, the proteins of the complex can be cross-linked to enhance the stability of the complex. Different methods to cross-link proteins are well known in the art. Reactive end-groups of cross-linking agents include but are not limited to —COOH, —SH, —NH2 or N-oxy-succinamate. The spacer of the cross-linking agent should be chosen with respect to the size of the complex to be cross-linked. For small protein complexes, comprising only a few proteins, relatively short spacers are preferable in order to reduce the likelihood of cross-linking separate complexes in the reaction mixture. For larger protein complexes, additional use of larger spacers is preferable in order to facilitate cross-linking between proteins within the complex.

It is preferable to check the success-rate of cross-linking before linking the complex to the carrier.

As will be apparent to a person skilled in the art, the optimal rate of cross-linking need to be determined on a case by case basis. This can be achieved by methods well known in the art, some of which are exemplary described below.

A sufficient rate of cross-linking can be checked f.e. by analysing the cross-linked complex vs. a non-cross-linked complex on a denaturating Protein-Gel. If cross-linking has been performed successfully, the proteins of the complex are expected to be found in the same lane, whereas the proteins of the non-cross-linked complex are expected to be separated according to their individual characteristics. Optionally the presence of all proteins of the complex can be further checked by peptide-sequencing of proteins in the respective bands using methods well known in the art such as Mass-spectrometry and/or Edman degradation.

In addition, a rate of crosslinking which is too high should also be avoided. If cross-linking has been carried out too extensively, there will be an increasing amount of cross-linking of the individual protein complex, which potentially interferes with a screening for potential binding partners and/or modulators etc. using the arrays. The presence of such structures can be determined by methods well known in the art and include f.e. gel-filtration experiments comparing the gel filtration profile solutions containing cross-linked complexes vs. uncross-linked complexes.

Optionally, functional assays as will be apparent to a person skilled in the art, some of which are exemplarily provided herein, can be performed to check the integrity of the complex.

Alternatively, members of the protein can be expressed as a single fusion protein and coupled to the matrix as will be apparent to a person skilled in the art.

Optionally, the attachment of the complex or proteins or antibody as outlined above can be further monitored by various methods apparent to a person skilled in the art. Those include, but are not limited to surface plasmon resonance (see f.e. McDonnel J M (2001) Curr Opin Chem Biol 5: 572-7; Lee K H (2001) Trends Biotechnol 19: 217-22; Weinberger S R, Morris T S, Pawlak M (2000) 1 (395-416; Pearson J E, Gill A, Vadagma P (2000) Ann Clin Biochem 37: 11945; Vely F, Trautmann A, Vivier E (2000) Methods Mol Biol 121: 313-21; Slepak V Z (2000) J. Mol Recognti 13: 20-6)

Moreover, a number of assays measuring the biochemical activity of the complex are described in the prior art

Exemplary assays useful for measuring the gamma-secretase activity of the protein complexes provided herein include but are not limited to those described in Li Y M et al (2000) Proc Natl Acad Sci USA 97: 6138-43; Pinnix I et al. (2001) J Biol Chem 276: 481-7; Karlstrom H et al. (2002) J Biol Chem 277: 6763-6

### 4.6.1. Candidate Molecules

Any molecule known in the art can be tested for its ability to modulate (increase or decrease) the amount of, activity of, or protein component composition of a complex of the present invention as detected by a change in the amount of, activity of, or protein component composition of, said complex. By way of example, a change in the amount of the complex can be detected by detecting a change in the amount of the complex that can be isolated from a cell expressing the complex machinery. For identifying a molecule that modulates complex activity, candidate molecules can be directly provided to a cell expressing the complex machinery, or, in the case of candidate proteins, can be provided by providing their encoding nucleic acids under conditions in which the nucleic acids are recombinantly expressed to produce the candidate proteins within the cell expressing the complex machinery, the complex is then isolated from the cell and the isolated complex is assayed for activity using methods well known in the art, not limited to those described, supra.

This embodiment of the invention is well suited to screen chemical libraries for molecules which modulate, e.g., inhibit, antagonize, or agonize, the amount of, activity of, or protein component composition of the complex. The chemical libraries can be peptide libraries, peptidomimetic libraries, chemically synthesized libraries, recombinant, e.g., phage display libraries, and in vitro translation-based libraries, other non-peptide synthetic organic libraries, etc.

Exemplary libraries are commercially available from several sources (ArQule, Tripos/PanLabs, ChemDesign, Pharmacopoeia). In some cases, these chemical libraries are generated using combinatorial strategies that encode the identity of each member of the library on a substrate to which the member compound is attached, thus allowing direct and immediate identification of a molecule that is an effective modulator. Thus, in many combinatorial approaches, the position on a plate of a compound specifies that compound's composition. Also, in one example, a single plate position may have from 1-20 chemicals that can be screened by administration to a well containing the interactions of interest. Thus, if modulation is detected, smaller and smaller pools of interacting pairs can be assayed for the modulation activity. By such methods, many candidate molecules can be screened.

Many diversity libraries suitable for use are known in the art and can be used to provide compounds to be tested according to the present invention. Alternatively, libraries can be constructed using standard methods. Chemical (synthetic) libraries, recombinant expression libraries, or polysome-based libraries are exemplary types of libraries that can be used.

The libraries can be constrained or semirigid (having some degree of structural rigidity), or linear or nonconstrained. The library can be a cDNA or genomic expression library, random peptide expression library or a chemically synthesized random peptide library, or non-peptide library. Expression libraries are introduced into the cells in which the assay occurs, where the nucleic acids of the library are expressed to produce their encoded proteins.

In one embodiment, peptide libraries that can be used in the present invention may be libraries that are chemically synthesized in vitro. Examples of such libraries are given in Houghten et al., 1991, Nature 354:84-86, which describes mixtures of free hexapeptides in which the first and second residues in each peptide were individually and specifically defined; Lam et al., 1991, Nature 354:82-84, which describes a “one bead, one peptide” approach in which a solid phase split synthesis scheme produced a library of peptides in which each bead in the collection had immobilized thereon a single, random sequence of amino acid residues; Medynski, 1994, Bio/Technology 12:709-710, which describes split synthesis and T-bag synthesis methods; and Gallop et al., 1994, J. Medicinal Chemistry 37(9):1233-1251. Simply by way of other examples, a combinatorial library may be prepared for use, according to the methods of Ohlmeyer et al., 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; or Salmon et al., 1993, Proc. Natl. Acad. Sci. USA 90:11708-11712. PCT Publication No. WO 93/20242 and Brenner and Lemer, 1992, Proc. Natl. Acad. Sci. USA 89:5381-5383 describe “encoded combinatorial chemical libraries,” that contain oligonucleotide identifiers for each chemical polymer library member.

In a preferred embodiment, the library screened is a biological expression library that is a random peptide phage display library, where the random peptides are constrained (e.g., by virtue of having disulfide bonding).

Further, more general, structurally constrained, organic diversity (e.g., nonpeptide) libraries, can also be used. By way of example, a benzodiazepine library (see e.g., Bunin et al., 1994, Proc. Natl. Acad. Sci. USA 91:4708-4712) may be used.

Conformationally constrained libraries that can be used include but are not limited to those containing invariant cysteine residues which, in an oxidizing environment, cross-link by disulfide bonds to form cystines, modified peptides (e.g., incorporating fluorine, metals, isotopic labels, are phosphorylated, etc.), peptides containing one or more non-naturally occurring amino acids, non-peptide structures, and peptides containing a significant fraction of—carboxyglutamic acid.

Libraries of non-peptides, e.g., peptide derivatives (for example, that contain one or more non-naturally occurring amino acids) can also be used. One example of these are peptoid libraries (Simon et al., 1992, Proc. Natl. Acad. Sci. USA 89:9367-9371). Peptoids are polymers of non-natural amino acids that have naturally occurring side chains attached not to the alpha carbon but to the backbone amino nitrogen. Since peptoids are not easily degraded by human digestive enzymes, they are advantageously more easily adaptable to drug use. Another example of a library that can be used, in which the amide functionalities in peptides have been permethylated to generate a chemically transformed combinatorial library, is described by Ostresh et al., 1994, Proc. Natl. Acad. Sci. USA 91:11138-11142).

The members of the peptide libraries that can be screened according to the invention are not limited to containing the 20 naturally occurring amino acids. In particular, chemically synthesized libraries and polysome based libraries allow the use of amino acids in addition to the 20 naturally occurring amino acids (by their inclusion in the precursor pool of amino acids used in library production). In specific embodiments, the library members contain one or more non-natural or non-classical amino acids or cyclic peptides. Non-classical amino acids include but are not limited to the D-isomers of the common amino acids, -amino isobutyric acid, 4-aminobutyric acid, Abu, 2-amino butyric acid;. -Abu, -Ahx, 6-amino hexanoic acid; Aib, 2-amino isobutyric acid; 3-amino propionic acid; omithine; norleucine; norvaline, hydroxyproline, sarcosine, citrulline, cysteic acid, t-butylglycine, t-butylalanine, phenylglycine, cyclohexylalanine, β-alanine, designer amino acids such as β-methyl amino acids, C-methyl amino acids, N-methyl amino acids, fluoro-amino acids and amino acid analogs in general. Furthermore, the amino acid can be D (dextrorotary) or L (levorotary).

In a specific embodiment, fragments and/or analogs of complexes of the invention, or protein components thereof, especially peptidomimetics, are screened for activity as competitive or non-competitive inhibitors of complex activity or formation.

In another embodiment of the present invention, combinatorial chemistry can be used to identify modulators of a the complexes. Combinatorial chemistry is capable of creating libraries containing hundreds of thousands of compounds, many of which may be structurally similar. While high throughput screening programs are capable of screening these vast libraries for affinity for known targets, new approaches have been developed that achieve libraries of smaller dimension but which provide maximum chemical diversity. (See e.g., Matter, 1997, Journal of Medicinal Chemistry 40:1219-1229).

One method of combinatorial chemistry, affinity fingerprinting, has previously been used to test a discrete library of small molecules for binding affinities for a defined panel of proteins. The fingerprints obtained by the screen are used to predict the affinity of the individual library members for other proteins or receptors of interest (in the instant invention, the protein complexes of the present invention and protein components thereof.) The fingerprints are compared with fingerprints obtained from other compounds known to react with the protein of interest to predict whether the library compound might similarly react. For example, rather than testing every ligand in a large library for interaction with a complex or protein component, only those ligands having a fingerprint similar to other compounds known to have that activity could be tested. (See, e.g., Kauvar et al., 1995, Chemistry and Biology 2:107-118; Kauvar, 1995, Affinity fingerprinting, Pharmaceutical Manufacturing International. 8:25-28; and Kauvar, Toxic-Chemical Detection by Pattern Recognition in New Frontiers in Agrochemical Immunoassay, D. Kurtz. L. Stanker and J. H. Skerritt. Editors, 1995, AOAC: Washington, D.C., 305-312).

Kay et al., 1993, Gene 128:59-65 (Kay) discloses a method of constructing peptide libraries that encode peptides of totally random sequence that are longer than those of any prior conventional libraries. The libraries disclosed in Kay encode totally synthetic random peptides of greater than about 20 amino acids in length. Such libraries can be advantageously screened to identify complex modulators. (See also U.S. Pat. No. 5,498,538 dated Mar. 12, 1996; and PCT Publication No. WO 94/18318 dated Aug. 18, 1994).

A comprehensive review of various types of peptide libraries can be found in Gallop et al., 1994, J. Med. Chem. 37:1233-1251.

### 4.7. Pharmaceutical Compositions and Therapeutic/Prophylactic Administration

The invention provides methods of treatment (and prophylaxis) by administration to a subject of an effective amount of a Therapeutic of the invention. In a preferred aspect, the Therapeutic is substantially purified. The subject is preferably an animal including, but not limited to animals such as cows, pigs, horses, chickens, cats, dogs, etc., and is preferably a mammal, and most preferably human. In a specific embodiment, a non-human mammal is the subject.

Various delivery systems are known and can be used to administer a Therapeutic of the invention, e.g., encapsulation in liposomes, microparticles, and microcapsules: use of recombinant cells capable of expressing the Therapeutic, use of receptor-mediated endocytosis (e.g., Wu and Wu, 1987, J. Biol. Chem. 262:4429-4432); construction of a Therapeutic nucleic acid as part of a retroviral or other vector, etc. Methods of introduction include but are not limited to intradermal, intramuscular, intraperitoneal, intravenous, subcutaneous, intranasal, epidural, and oral routes. The compounds may be administered by any convenient route, for example by infusion, by bolus injection, by absorption through epithelial or mucocutaneous linings (e.g., oral, rectal and intestinal mucosa, etc.), and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment. This may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

In another embodiment, the Therapeutic can be delivered in a vesicle, in particular a liposome (Langer, 1990, Science 249:1527-1533; Treat et al., 1989, In: Liposomes in the Therapy of Infectious Disease and Cancer, Lopez-Berestein and Fidler, eds., Liss, N.Y., pp. 353-365; Lopez-Berestein, ibid., pp. 317-327; see generally ibid.)

In yet another embodiment, the Therapeutic can be delivered via a controlled release system. In one embodiment, a pump may be used (Langer, supra; Sefton, 1987, CRC Crit. Ref. Biomed. Eng. 14:201-240; Buchwald et al., 1980, Surgery 88:507-516; Saudek et al., 1989, N. Engl. J. Med. 321:574-579). In another embodiment, polymeric materials can be used (Medical Applications of Controlled Release, Langer and Wise, eds., CRC Press, Boca Raton, Fla., 1974; Controlled Drug Bioavailability, Drug Product Design and Performance, Smolen and Ball, eds., Wiley, New York, 1984; Ranger and Peppas, 1983, Macromol. Sci. Rev. Macromol. Chem. 23:61; Levy et al., 1985, Science 228:190-192; During et al., 1989, Ann. Neurol. 25:351-356; Howard et al., 1989, J. Neurosurg. 71:858-863). In yet another embodiment, a controlled release system can be placed in proximity of the therapeutic target, i.e., the brain, thus requiring only a fraction of the systemic dose (e.g., Goodson, 1984, In: Medical Applications of Controlled Release, supra, Vol. 2, pp.115-138). Other controlled release systems are discussed in the review by Langer (1990, Science 249:1527-1533).

In a specific embodiment where the Therapeutic is a nucleic acid encoding a protein Therapeutic, the nucleic acid can be administered in vivo to promote expression of its encoded protein, by constructing it as part of an appropriate nucleic acid expression vector and administering it so that it becomes intracellular, e.g., by use of a retroviral vector (U.S. Pat. No. 4,980,286), or by direct injection, or by use of microparticle bombardment (e.g., a gene gun; Biolistic, Dupont), or by coating it with lipids, cell-surface receptors or transfecting agents, or by administering it in linkage to a homeobox-like peptide which is known to enter the nucleus (e.g., Joliot et al., 1991, Proc. Natl. Acad. Sci. USA 88:1864-1868), etc. Alternatively, a nucleic acid Therapeutic can be introduced intracellularly and incorporated by homologous recombination within host cell DNA for expression.

The present invention also provides pharmaceutical compositions. Such compositions comprise a therapeutically effective amount of a Therapeutic, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water is a preferred carrier when the pharmaceutical composition is administered orally. Saline and aqueous dextrose are preferred carriers when the pharmaceutical composition is administered intravenously. Saline solutions and aqueous dextrose and glycerol solutions are preferably employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the Therapeutic, preferably in purified form, together with a suitable amount of carrier so as to provide the form for proper administration to the patient. The formulation should suit the mode of administration.

In a preferred embodiment, the composition is formulated, in accordance with routine procedures, as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water or saline for injection can be provided so that the ingredients may be mixed prior to administration.

The Therapeutics of the invention can be formulated as neutral or salt forms. Pharmaceutically acceptable salts include those formed with free carboxyl groups such as those derived from hydrochloric, phosphoric, acetic, oxalic, tartaric acids, etc., those formed with free amine groups such as those derived from isopropylamine, triethylamine, 2-ethylamino ethanol, histidine, procaine, etc., and those derived from sodium, potassium, ammonium, calcium, and ferric hydroxides, etc.

The amount of the Therapeutic of the invention which will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The invention also provides a pharmaceutical pack or kit comprising one or more containers filled with one or more of the ingredients of the pharmaceutical compositions of the invention. For example, the kit can comprise in one container Sambiasin-1 or Sambiasin-2 or a functionally active fragment or functionally active derivative of said proteins and in at least one other container any of the proteins Nicastrin or Presenilin-1 or Presenilin-2 or a functionally active fragment or functionally active derivative of said proteins.

The kits of the present invention can also contain expression vectors encoding the essential components of the complex machinery, which components after being expressed can be reconstituted in order to form a biologically active complex. Such a kit preferably also contains the required buffers and reagents. Optionally associated with such container(s) can be instructions for use of the kit and/or a notice in the form prescribed by a governmental agency regulating the manufacture, use or sale of pharmaceuticals or biological products, which notice reflects approval by the agency of manufacture, use or sale for human administration.

### 4.8 Animal Models

The present invention also provides animal models. In one embodiment, animal models for diseases and disorders involving the protein complexes of the present invention are provided. These animal models are well known in the art. These animal models include, but are not limited to those which are listed in the section 4.6 as exemplary animald models to study any of the complexes provided in the invention. Such animals can be initially produced by promoting homologous recombination or insertional mutagenesis between genes encoding the protein components of the complexes in the chromosome, and exogenous genes encoding the protein components of the complexes that have been rendered biologically inactive or deleted (preferably by insertion of a heterologous sequence, e.g., an antibiotic resistance gene). In a preferred aspect, homologous recombination is carried out by transforming embryo-derived stem (ES) cells with one or more vectors containing one or more insertionally inactivated genes, such that homologous recombination occurs, followed by injecting the transformed ES cells into a blastocyst, and implanting the blastocyst into a foster mother, followed by the birth of the chimeric animal (“knockout animal”) in which a gene encoding either Sambiasin-1 or Sambiasin-2 or a functionally active fragment or functionally active derivative of said proteins and a gene encoding either Nicastrin or Presenilin-1 or Presenilin-2 , or a functionally active fragment or functionally active derivative of said proteins, has been inactivated or deleted (Capecchi, 1989, Science 244:1288-1292).

The chimeric animal can be bred to produce additional knockout animals. Such animals can be mice, hamsters, sheep, pigs, cattle, etc., and are preferably non-human mammals. In a specific embodiment, a knockout mouse is produced.

Such knockout animals are expected to develop, or be predisposed to developing, diseases or disorders associated with mutations involving the protein complexes of the present invention, and thus, can have use as animal models of such diseases and disorders, e.g., to screen for or test molecules (e.g., potential Therapeutics) for such diseases and disorders.

In a different embodiment of the invention, transgenic animals that have incorporated and express (or over-express or mis-express) a functional gene encoding a protein component of the complex, e.g. by introducing the a gene encoding one or more of the components of the complex under the control of a heterologous promoter (i.e., a promoter that is not the native promoter of the gene) that either over-expresses the protein or proteins, or expresses them in tissues not normally expressing the complexes or proteins, can have use as animal models of diseases and disorders characterized by elevated levels of the protein complexes. Such animals can be used to screen or test molecules for the ability to treat or prevent the diseases and disorders cited supra.

In one embodiment, the present invention provides a recombinant non-human animal in which an endogenous gene encoding a first protein, or a functionally active fragment or functionally active derivative thereof, which first protein is selected from the group of proteins of Sambiasin-1 and Sambiasin-2, and and endogenous gene encoding a second protein, or a functionally active fragment or functionally active derivative thereof, which second protein is selected from the group consisting of proteins of Nicastrin and Presenilin-1 and Presenilin-2 has been deleted or inactivated by homologous recombination or insertional mutagenesis of said animal or an ancestor thereof. In addition, the present invention provides a recombinant non-human animal in which the endogenous gene of Sambiasin-1 has been inactivated.

In another embodiment, the present invention provides a recombinant non-human animal in which an endogenous gene encoding a first protein, or a functionally active fragment or functionally active derivative thereof, which first protein is selected from the group consisting of proteins of Sambiasin-1 and Sambiasin-2, and endogenous gene encoding a second protein, or a functionally active fragment or functionally active derivative thereof, which second protein is selected from the group consisting of proteins of Nicastrin and Presenilin-1 and Presenilin-2 are recombinantly expressed in said animal or an ancestor thereof.

The following series of examples are presented by way of illustration and not by way of limitation on the scope of the invention.

### 5. EXAMPLES

### Isolation of the Protein Complexes/Protein of the Invention from Mouse

2 Mouse forebrains (0.6314 g total wet weight) were lysed in 14 mls of 50 mM HEPES pH7.4; 150 mM NaCl; 1 mM EDTA; 0.5 mM Sodium Vanadate; 10% Glycerol; 1% Dodecyl maltoside containing standard proteinase inhibitors. The tissue was homogenised in a Warring blender for 30 seconds on ice. Homogenates were incubated on ice for 1 hour & then centrifuged at 13,000 g for 30 min at 40° C. The resulting pellet was stored at −80° C. while the supernatant was centrifuged at 50,000 g for 30 min at 4° C. and the pellet from this second centrifugation step was also stored at −80° C. 6.5 ml of the supernatant from this second centrifugation step was taken and combined with 25 μl of anti presenilin-1 antisera (MAB5232, Chemicon). The antibody/lysate mixture was incubated for 1 hour at 4° C. with end-over end mixing. Pre-washed protein G sepharose was added and the mixture incubated overnight at 4° C. with end-over mixing. The protein G was recovered by centrifugation at 200 g for 5 min at 4° C. The protein G beads were then washed 5 times in 1 ml lysis buffer (containing 0.1% DDM rather than 1%). 100 μl of NuPAGE sample buffer (Invitrogen) was added and the sample incubated at 37° C. for 10 min. Samples were separated on 4-12% NuPAGE bis/tris gels (Invitrogen). Proteins were visualized by staining with colloidal coomassie (Sigma) & then analysed by LC/MSMS.

### Identification of the Proteins of the Invention

Gel-separated proteins were reduced, alkylated and digested in gel essentially following the procedure described by Shevchenko et al. (Shevchenko, A., Wilm, M., Vorm, O., Mann, M. Anal Chem 1996, 68, 850-858). Briefly, gel-separated proteins were excised from the gel using a clean scalpel, reduced using 10 mM DTT (in 5 mM ammonium bicarbonate, 54° C., 45 min) and subsequently alkylated with 55 mM iodoacetamid (in 5 mM ammonium bicarbonate) at room temperature in the dark (30 min). Reduced and alkylated proteins were digested in gel with porcine trypsin (Promega) at a protease concentration of 12.5 ng/μl in 5 mM ammonium bicarbonate. Digestion was allowed to proceed for 4 hours at 37 ° C. and the reaction was subsequently stopped using 5 μl 5% formic acid.

Gel plugs were extracted twice with 20 μl 1% TFA and pooled with acidified digest supernatants. Samples were dried in a a vaccum centrifuge and resuspended in 13 μl 1% TFA.

Peptide samples were injected into a nano LC system (CapLC, Waters or Ultimate, Dionex) which was directly coupled either to a quadrupole TOF (QTOF2, QTOF Ultima, QTOF Micro, Micromass or QSTAR Pulsar, Sciex) or ion trap (LCQ Deca XP) mass spectrometer. Peptides were separated on the LC system using a gradient of aqueous and organic solvents (see below). Solvent A was 5% acetonitrile in 0.5% formic acid and

| Time (min) | % solvent A | % solvent B |
|---|---|---|
| 0 | 95 | 5 |
| 5.33 | 92 | 8 |
| 35 | 50 | 50 |
| 36 | 20 | 80 |
| 40 | 20 | 80 |
| 41 | 95 | 5 |
| 50 | 95 | 5 |

Peptides eluting off the LC system were partially sequenced within the mass spectrometer.

The peptide mass and fragmentation data generated in the LC-MS/MS experiments were used to query fasta formatted protein and nucleotide sequence databases maintained and updated regularly at the NCBI (for the NCBInr, dbEST and the human and mouse genomes) and European Bioinformatics Institute (EBI, for the human, mouse, Drosophila and C. Elegans proteome databases). Proteins were identified by correlating the measured peptide mass and fragmentation data with the same data computed from the entries in the database using the software tool Mascot (Matrix Science, Perkins, D. N., Pappin, D. J., Creasy, D. M., Cottrell, J. S., Electrophoresis 1999, 20, 3551-67). Search criteria varied depending on which mass spectrometer was used for the analysis.

Exemplary assays useful for measuring the gamma-secretase activity of the protein complexes provided herein include but are not limited to those described in Li Y M et al (2000) Proc Natl Acad Sci USA 97: 6138-43; Pinnix I et al. (2001) J Biol Chem 276: 481-7; Karlstrom H et al. (2002) J Biol Chem 277: 6763-6

### Sequences

(Sambiasin-1)

SEQ ID No: 1

MGAAVFFGCTFVAFGPAFALFLITVAGDPLRVIILVAGAFFWLVSLLLAS
VVWFILVHVTDRSDARLQYGLLIFGAAVSVLLQEVFRFAYYKLLKKADEG
LASLSEDGRSPISIRQMAYVSGLSFGIISGVFSVINILADALGPGVVGIH
GDSPYYFLTSAFLTAAIILLHTFWGWFFDACERRRYWALGLWGSHLLTSG
LTFLNPWYEASLLPIYAVTVSMGLWAFITAGGSLRSIQRSLLCKD
(Presenilin-1)

SEQ ID No: 2

MTELPAPLSY FQNAQMSEDN HLSNTVRSQN DNRERQEHND
RRSLGHPEPL SNGRPQGNSR QVVEQDEEED EELTLKYGAK
HVIMLFVPVT LCMVVVVATI KSVSFYTRKD GQLIYTPFTE
DTETVGQRAL HSILNAAIMI SVIVVMTILL VVLYKYRCYK
VIHAWLIISS LLLLFFFSFI YLGEVFKTYN VAVDYITVAL
LIWNFGVVGM ISIHWKGPLR LQQAYLIMIS ALMALVFIKY
LPEWTAWLIL AVISVYDLVA VLCPKGPLRM LVETAQERNE
TLFPALIYSS TMVWLVNMAE GDPEAQRRVS KNSKYNAEST
ERESQDTVAE NDDGGFSEEW EAQRDSHLGP HRSTPESRAA
VQELSSSILA GEDPEERGVK LGLGDFIFYS VLVGKASATA
SGDWNTTIAC FVAILIGLCL TLLLLAIFKK ALPALPISIT
FGLVFYFATD YLVQPFMDQL AFHQFYI
(Nicastrin)

SEQ ID No: 3

MATAGGGSGA DPGSRGLLRL LSFCVLLAGL CRGNSVERKI
YIPLNKTAPC VRLLNATHQI GCQSSISGDT GVIHVVEKEE
DLQWVLTDGP NPPYMVLLES KHFTRDLMEK LKGRTSRIAG
LAVSLTKPSP ASGFSPSVQC PNDGFGVYSN SYGPEFAHCR
EIQWNSLGNG LAYEDFSFPI FLLEDENETK VIKQCYQDHN
LSQNGSAPTF PLCAMQLFSH MHAVISTATC MRRSSIQSTF
SINPEIVCDP LSDYNVWSML KPINTTGTLK PDDRVVVAAT
RLDSRSFFWN VAPGAESAVA SFVTQLAAAE ALQKAPDVTT
LPRNVMFVFF QGETFDYIGS SRMVYDMEKG KFPVQLENVD
SFVELGQVAL RTSLELWMHT DPVSQKNESV RNQVEDLLAT
LEKSGAGVPA VILRRPNQSQ PLPPSSLQRF LRARNISGVV
LADHSGAFHN KYYQSIYDTA ENINVSYPEW LSPEEDLNFV
TDTAKALADV ATVLGRALYE LAGGTNFSDT VQADPQTVTR
LLYGFLIKAN NSWFQSILRQ DLRSYLGDGP LQHYIAVSSP
TNTTYVVQYA LANLTGTVVN LTREQCQDPS KVPSENKDLY
EYSWVQGPLH SNETDRLPRC VRSTARLARA LSPAFELSQW
SSTEYSTWTE SRWKDIRARI FLIASKELEL ITLTVGFGIL
IFSLIVTYCI NAKADVLFIA PREPGAVSY
(Sambiasin-2)

SEQ ID No: 4

MTAAVFFGCA FIAFGPALAL YVFTIATEPL RIIFLIAGAF
FWLVSLLISS LVWFMARVIIDNKDGPTQKY LLIFGAFVSV
YIQEMFRFAY YKLLKKASEG LKSINPGETA
PSMRLLAYVSGLGFGIMSGV FSFVNTLSDS LGPGTVGIHG
DSPQFFLYSA FMTLVIILLH VFWGIVFFDGCEKKKWGILL
IVLLTHLLVS AQTFISSYYG INLASAFIIL VLMGTWAFLA
AGGSCRSLKLCLLCQDKNFL LYNQRSR
(Presenilin-2)

SEQ ID No: 5

MLTFMASDSE EEVCDERTSL MSAESPTPRS CQEGRQGPED
GENTAQWRSQ ENEEDGEEDP DRYVCSGVPG RPPGLEEELT
LKYGAKHVIM LFVPVTLCMI VVVATIKSVR FYTEKNGQLI
YTPFTEDTPS VGQRLLNSVL NTLIMISVIV VMTIFLVVLY
KYRCYKFIHG WLIMSSLMLL FLFTYIYLGE VLKTYNVAMD
YPTLLLTVWN FGAVGMVCIH WKGPLVLQQA YLIMISALMA
LVFIKYLPEW SAWVILGAIS VYDLVAVLCP KGPLRMLVET
AQERNEPIFP ALIYSSAMVW TVGMAKLDPS SQGALQLPYD
PEMEEDSYDS FGEPSYPEVF EPPLTGYPGE ELEEEEERGV
KLGLGDFIFY SVLVGKAAAT GSGDWNTTLA CFVAILIGLC
LTLLLLAVFK KALPALPISI TFGLIFYFST DNLVRPFMDT
LASHQLYI
(GenBank AAD34072)

SEQ ID No: 6

MGAAVFFGCTFVAFGPAFALFLITVAGDPLRVIILVAGAFFWLVSLLLAS
VVWFILVHVTDRSDARLQYGLLIFGAAVSVLLQEVFRFAYYKLLKKADEG
LASLSEDGRSPISIRQMAYVSGLSFGIISGVFSVINILADALGPGVVGIH
GDSPYYFLTSAFLTAAIILLHTFWGVVFFDACERRRYWALGLVVGSHLLT
SGLTFLNPWYEASLLPIYAVTVSMGLWAFITAGGSLRSIQRSSCVRTDYL
D

1. A protein complex comprising
(a) a first protein, or a functionally active fragment or functionally active derivative thereof, which first protein is selected from the group consisting of:
(i) Sambiasin-1 (SEQ ID No: 1) or a functionally active derivative thereof, or a functionally active fragment thereof, or a homolog thereof, or a variant of Sambiasin-1 encoded by a nucleic acid that hybridizes to the Sambiasin-1 nucleic acid or its complement under low stringency conditions,
(b) and at least one second protein, or a functionally active fragment or functionally active derivative thereof, which second protein is selected from the group consisting of:
(i) Presenilin-1 (SEQ ID No: 2), or a functionally active derivative thereof, or a functionally active fragment thereof, or a homolog thereof, or a variant of Presenilin-1 encoded by a nucleic acid that hybridizes to the Presenilin-1 nucleic acid or its complement under low stringency conditions,
(ii) Nicastrin (SEQ ID No: 3), or a functionally active derivative thereof, or a functionally active fragment thereof, or a homolog thereof, or a variant of Nicastrin encoded by a nucleic acid that hybridizes to the Nicastrin nucleic acid or its complement under low stringency conditions, wherein said first protein and said second protein are members of a native cellular complex, and wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.

2. A protein complex comprising Sambiasin-1 (SEQ ID No: 1) or Sambiasin-2 (SEQ ID No: 4) and Presenilin-1 (SEQ ID No: 2) or Presenilin-2 (SEQ ID No: 5).

3. A protein complex according to claim 1 further comprising Nicastrin (SEQ ID No: 3)

4. A protein complex according to claim 1 comprising Sambiasin-1 (SEQ ID No: 1) and Presenilin-1 (SEQ ID No: 2) and Nicastrin (SEQ ID No: 3).

5. The complex of claim 1 comprising a functionally active derivative of any of the proteins of said complex, wherein the functionally active derivative is a fusion protein comprising said protein fused to an amino acid sequence different from said protein.

6. The complex of claim 5 wherein the functionally active derivative is a fusion protein comprising said protein fused to an affinity tag or label.

7. The complex of claim 1 comprising a fragment of any of the proteins of said complex, which fragment binds to another protein component of said complex.

8. The complex of claim 1 that is involved in the gamma-secretase activity.

9. A protein comprising the amino acid sequence of SEQ ID No: 1, or a functionally active derivative thereof, or a functionally active fragment thereof, or a homolog thereof, or a variant of Sambiasin-1 encoded by a nucleic acid that hybridizes to the Sambiasin-1 nucleic acid or its complement under low stringency conditions, wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C., with the proviso that the protein does not have the amino acid sequence according to SEQ ID No: 6.

10. Protein comprising the amino acid sequence of SEQ ID No: 1.

11. A nucleic acid encoding a protein according to claim 9.

12. A construct comprising
(a) a nucleic acid according to claim 11 and at least one further nucleic acid which is normally not associated with said nucleic acid, or
(b) at least two separate nucleic acid sequences each encoding a different protein, which protein is selected from the group consisting of Sambiasin-1 (SEQ ID No: 1), Presenilin-1 (SEQ ID No: 2), and Nicastrin (SEQ ID No: 3), or a functionally active fragment or a functionally active derivative thereof.

13. A host cell containing one or more constructs according to claim 12.

14. An antibody or a fragment of said antibody containing the binding domain thereof, which binds the complex of claim 1, and which does not bind the first protein when uncomplexed or the second protein when uncomplexed.

15. A kit comprising in one or more containers the complex of claim 1 optionally together with reagents and working instructions.

16. A kit according to claim 15 for the diagnosis or prognosis of a disease or a disease risk, wherein the disease is selected from the group consisting of neurodegenerative diseases and developmental disorders caused by defects in the Notch pathway.

17. An array comprising the complex according to claim 1 attached to a solid carrier.

18. A method for processing the physiological substrates of the complex of claim 1 comprising the step of bringing into contact the complex with said substrate, such that said substrate is processed.

19. A pharmaceutical composition comprising the protein complex of claim 1 and a pharmaceutically acceptable carrier.

20. A pharmaceutical composition according to claim 19 for the treatment of diseases and disorders, wherein the disease or disorder is selected from the group consisting of neurodegenerative diseases and developmental disorders caused by defects in the Notch pathway.

21. A method for screening for a molecule that binds to the complex of claim 1, comprising the following steps:
(a) exposing said complex, or a cell or organism containing same, to one or more candidate molecules; and
(b) determining whether said candidate molecule is bound to the complex.

22. A method for screening for a molecule that modulates the function, activity, composition or formation of the complex of claim 1 comprising the steps of:
(a) exposing said complex, or a cell or organism containing said complex to one or more candidate molecules; and
(b) determining the amount of, activity of, protein components of, and/or intracellular localization of, said complex and/or the transcription level of a gene dependent on the complex and/or the abundance and/or activity of a protein or protein complex dependent on the function of the complex and/or product of a gene dependent on the complex in the presence of the one or more candidate molecules, wherein a change in said amount, activity, protein components or intracellular localization relative to said amount, activity, protein components and/or intracellular localization and/or a change in the transcription level of a gene dependent on the complex and/or the abundance and/or activity of a protein or protein complex dependent on the function of the complex and/or product of a gene dependent on the complex in the absence of said candidate molecules indicates that the molecule modulates function, activity or composition of said complex.

23. The method of claim 22 wherein the amount of said complex is determined.

24. The method of claim 22, wherein the activity of said complex is determined.

25. The method of claim 24, wherein said determining step comprises isolating from the cell or organism said complex to produce said isolated complex and contacting said isolated complex in the presence or absence of a candidate molecule with a physiological substrate of the complex and determine whether said substrate is processed.

26. The method of claim 22, wherein the amount of the individual protein components of said complex are determined.

27. The method of claim 26, wherein said determining step comprises determining whether
(i) Sambiasin-1 (SEQ ID No: 1) or a functionally active derivative thereof, or a functionally active fragment thereof, or a homolog thereof, or a variant of Sambiasin-1 encoded by a nucleic acid that hybridizes to the Sambiasin-1 nucleic acid or its complement under low stringency conditions, and/or
(ii) Presenilin-1 (SEQ ID No: 2), or a functionally active derivative thereof, or a functionally active fragment thereof, or a homolog thereof, or a variant of Presenilin-1 encoded by a nucleic acid that hybridizes to the Presenilin-1 nucleic acid or its complement under low stringency conditions, and/or
(iii) Nicastrin (SEQ ID No: 3), or a functionally active derivative thereof, or a functionally active fragment thereof, or a homolog thereof, or a variant of Nicastrin encoded by a nucleic acid that hybridizes to the Nicastrin nucleic acid or its complement under low stringency conditions,
are present in the complex and wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.

28. The method of claim 22, wherein said method is a method of screening for a drug for treatment or prevention of a disease or disorder, wherein the disease or disorder is selected from the group consisting of neurodegenerative diseases and developmental disorders caused by defects in the Notch pathway.

29. A method of manufacturing a medicament comprising the use of a molecule that modulates the amount of, activity of, or the protein components of the complex of claim 1 to manufacture a medicament for the treatment or prevention of a disease or disorder, wherein the disease or disorder is selected from the group consisting of neurodegenerative diseases and developmental disorders caused by defects in the Notch pathway.

30. A method for the production of a pharmaceutical composition comprising carrying out the method of claim 22 to identify a molecule that modulates the function, activity, composition or formation of said complex, and further comprising mixing the identified molecule with a pharmaceutically acceptable carrier.

31. A method for diagnosing or screening for the presence of a disease or disorder or a predisposition for developing a disease or disorder in a subject, which disease or disorder is characterized by an aberrant amount of, activity of, component composition of, or intracellular localization of the complex of claim 1 comprising determining the amount of, activity of, protein components of, and/or intracellular localization of, said complex and/or the transcription level of a gene dependent on the complex and/or the abundance and/or activity of a protein or protein complex dependent on the function of the complex and/or product of a gene dependent on the complex in a comparative sample derived from a subject, wherein a difference in said amount, activity, or protein components of, said complex in an analogous sample from a subject not having the disease or disorder or predisposition indicates the presence in the subject of the disease or disorder or predisposition in the subject.

32. The method of claim 31, wherein the amount of said complex is determined.

33. The method of claim 31, wherein the activity of said complex is determined.

34. The method of claim 33, wherein said determining step comprises isolating from the cell or organism said complex to produce said isolated complex and contacting said isolated complex in the presence or absence of a candidate molecule with a physiological substrate of the complex, and determine whether said substrate is processed.

35. The method of claim 31, wherein the amount of the individual protein components of said complex are determined.

36. The method of claim 35, wherein said determining step comprises determining whether
(i) Sambiasin-1 (SEQ ID No: 1) or a functionally active derivative thereof, or a functionally active fragment thereof, or a homolog thereof, or a variant of Sambiasin-1 encoded by a nucleic acid that hybridizes to the Sambiasin-1 nucleic acid or its complement under low stringency conditions, and/or
(ii) Presenilin-1 (SEQ ID No: 2), or a functionally active derivative thereof, or a functionally active fragment thereof, or a homolog thereof, or a variant of Presenilin-1 encoded by a nucleic acid that hybridizes to the Presenilin-1 nucleic acid or its complement under low stringency conditions, and/or
(iii) Nicastrin (SEQ ID No: 3), or a functionally active derivative thereof, or a functionally active fragment thereof, or a homolog thereof, or a variant of Nicastrin encoded by a nucleic acid that hybridizes to the Nicastrin nucleic acid or its complement under low stringency conditions,
are present in the complex and wherein said low stringency conditions comprise hybridization in a buffer comprising 35% formamide, 5×SSC, 50 mM Tris-HCl (pH 7.5), 5 mM EDTA, 0.02% PVP, 0.02% Ficoll, 0.2% BSA, 100 ug/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1.5 hours at 55° C., and washing in a buffer consisting of 2×SSC, 25 mM Tris-HCl (pH 7.4), 5 mM EDTA, and 0.1% SDS for 1.5 hours at 60° C.

37. The method of claim 31, wherein the disease or disorder is selected from the group consisting of neurodegenerative diseases and developmental disorders caused by defects in the Notch pathway.

38. A method for treating or preventing a disease or disorder characterized by an aberrant amount of, activity of, component composition of or intracellular localization of, the complex of claim 1 comprising administering to a subject in need of such treatment or prevention a therapeutically effective amount of one or more molecules that modulate the amount of, activity of, or protein components of, said complex.

39. The method according to claim 38, wherein said disease or disorder involves decreased levels of the amount or activity of said complex.

40. The method according to claim 39, wherein said disease or disorder involves increased levels of the amount or activity of said complex.

41. (canceled)

42. An antibody or a fragment of said antibody containing the binding domain thereof, which binds to the protein of claim 9.

43. A kit comprising in one or more containers the protein of claim 9, optionally together with reagents and working instructions.

44. The kit of claim 15, which further comprises a protein comprising the amino acid sequence of SEQ ID No: 1, or a functionally active derivative thereof, or a functionally active fragment thereof, or a homolog thereof

45. The kit of claim 15, which further comprises an antibody or a fragment of said antibody containing the binding domain thereof, which binds the complex.

46. An array comprising the protein of claim 9 attached to a solid carrier.

47. An array comprising at least one antibody according to claim 14 attached to a solid carrier.

48. A pharmaceutical composition comprising the protein of claim 9 and a pharmaceutically acceptable carrier.

49. A method for screening for a molecule that binds to the protein of claim 9 comprising the following steps:
(a) exposing said protein, or a cell or organism containing same, to one or more candidate molecules; and
(b) determining whether said candidate molecule is bound to the protein.


SEQUENCE LISTING (6 sequences)

SEQ ID NO: 1 - protein, 247 aa, Homo sapiens
MGAAVFFGCTFVAFGPAFALFLITVAGDPLRVIILVAGAFFWLVSLLLASVVWFILVHVTDRSDARLQYGLLIFGAAVSVLLQEVFRFAYYKLLKKADEGLASLSEDGRSPISIRQMAYVSGLSFGIISGVFSVINILADALGPGVVGIHGDSPYYFLTSAFLTAAIILLHTFWGVVFFDACERRRYWALGLVVGSHLLTSGLTFLNPWYEASLLPIYAVTVSMGLWAFITAGGSLRSIQRSLLCKD

SEQ ID NO: 2 - protein, 467 aa, Homo sapiens
MTELPAPLSYFQNAQMSEDNHLSNTVRSQNDNRERQEHNDRRSLGHPEPLSNGRPQGNSRQVVEQDEEEDEELTLKYGAKHVIMLFVPVTLCMVVVVATIKSVSFYTRKDGQLIYTPFTEDTETVGQRALHSILNAAIMISVIVVMTILLVVLYKYRCYKVIHAWLIISSLLLLFFFSFIYLGEVFKTYNVAVDYITVALLIWNFGVVGMISIHWKGPLRLQQAYLIMISALMALVFIKYLPEWTAWLILAVISVYDLVAVLCPKGPLRMLVETAQERNETLFPALIYSSTMVWLVNMAEGDPEAQRRVSKNSKYNAESTERESQDTVAENDDGGFSEEWEAQRDSHLGPHRSTPESRAAVQELSSSILAGEDPEERGVKLGLGDFIFYSVLVGKASATASGDWNTTIACFVAILIGLCLTLLLLAIFKKALPALPISITFGLVFYFATDYLVQPFMDQLAFHQFYI

SEQ ID NO: 3 - protein, 709 aa, Homo sapiens
MATAGGGSGADPGSRGLLRLLSFCVLLAGLCRGNSVERKIYIPLNKTAPCVRLLNATHQIGCQSSISGDTGVIHVVEKEEDLQWVLTDGPNPPYMVLLESKHFTRDLMEKLKGRTSRIAGLAVSLTKPSPASGFSPSVQCPNDGFGVYSNSYGPEFAHCREIQWNSLGNGLAYEDFSFPIFLLEDENETKVIKQCYQDHNLSQNGSAPTFPLCAMQLFSHMHAVISTATCMRRSSIQSTFSINPEIVCDPLSDYNVWSMLKPINTTGTLKPDDRVVVAATRLDSRSFFWNVAPGAESAVASFVTQLAAAEALQKAPDVTTLPRNVMFVFFQGETFDYIGSSRMVYDMEKGKFPVQLENVDSFVELGQVALRTSLELWMHTDPVSQKNESVRNQVEDLLATLEKSGAGVPAVILRRPNQSQPLPPSSLQRFLRARNISGVVLADHSGAFHNKYYQSIYDTAENINVSYPEWLSPEEDLNFVTDTAKALADVATVLGRALYELAGGTNFSDTVQADPQTVTRLLYGFLIKANNSWFQSILRQDLRSYLGDGPLQHYIAVSSPTNTTYVVQYALANLTGTVVNLTREQCQDPSKVPSENKDLYEYSWVQGPLHSNETDRLPRCVRSTARLARALSPAFELSQWSSTEYSTWTESRWKDIRARIFLIASKELELITLTVGFGILIFSLIVTYCINAKADVLFIAPREPGAVSY

SEQ ID NO: 4 - protein, 257 aa, Homo sapiens
MTAAVFFGCAFIAFGPALALYVFTIATEPLRIIFLIAGAFFWLVSLLISSLVWFMARVIIDNKDGPTQKYLLIFGAFVSVYIQEMFRFAYYKLLKKASEGLKSINPGETAPSMRLLAYVSGLGFGIMSGVFSFVNTLSDSLGPGTVGIHGDSPQFFLYSAFMTLVIILLHVFWGIVFFDGCEKKKWGILLIVLLTHLLVSAQTFISSYYGINLASAFIILVLMGTWAFLAAGGSCRSLKLCLLCQDKNFLLYNQRSR

SEQ ID NO: 5 - protein, 448 aa, Homo sapiens
MLTFMASDSEEEVCDERTSLMSAESPTPRSCQEGRQGPEDGENTAQWRSQENEEDGEEDPDRYVCSGVPGRPPGLEEELTLKYGAKHVIMLFVPVTLCMIVVVATIKSVRFYTEKNGQLIYTPFTEDTPSVGQRLLNSVLNTLIMISVIVVMTIFLVVLYKYRCYKFIHGWLIMSSLMLLFLFTYIYLGEVLKTYNVAMDYPTLLLTVWNFGAVGMVCIHWKGPLVLQQAYLIMISALMALVFIKYLPEWSAWVILGAISVYDLVAVLCPKGPLRMLVETAQERNEPIFPALIYSSAMVWTVGMAKLDPSSQGALQLPYDPEMEEDSYDSFGEPSYPEVFEPPLTGYPGEELEEEEERGVKLGLGDFIFYSVLVGKAAATGSGDWNTTLACFVAILIGLCLTLLLLAVFKKALPALPISITFGLIFYFSTDNLVRPFMDTLASHQLYI

SEQ ID NO: 6 - protein, 251 aa, Homo sapiens
MGAAVFFGCTFVAFGPAFALFLITVAGDPLRVIILVAGAFFWLVSLLLASVVWFILVHVTDRSDARLQYGLLIFGAAVSVLLQEVFRFAYYKLLKKADEGLASLSEDGRSPISIRQMAYVSGLSFGIISGVFSVINILADALGPGVVGIHGDSPYYFLTSAFLTAAIILLHTFWGVVFFDACERRRYWALGLVVGSHLLTSGLTFLNPWYEASLLPIYAVTVSMGLWAFITAGGSLRSIQRSSCVRTDYLD